(19) United States
(12) Patent Application Publication
(10) Pub. No.: US 2024/0018602 A1
(43) Pub. Date: Jan. 18, 2024

(54) POLYMORHPISM DETECTION FOR GLIOBLASTOMA DETECTION AND TREATMENT

(71) Applicant: The Cleveland Clinic Foundation, Cleveland, OH (US)

(72) Inventors: Justin Lathia, Shaker Heights, OH (US); Tyler Alban, Cleveland, OH (US); Defne Baylik, Cleveland Heights, OH (US); Michael Vogelbaum, Cleveland, OH (US); Balint Otvos, Cleveland, OH (US); Matthew Grabowski, Cleveland, OH (US)

(21) Appl. No.: 18/252,612

(22) PCT Filed: Nov. 11, 2021
(86) PCT No.: PCT/US21/58961

§ 371 Date: May 11, 2023

Related U.S. Application Data
(60) Provisional application No. 63/112,461, filed on Nov. 11, 2020.

(51) Int. Cl. C12Q 1/6886; C12Q 1/6869; G01N 33/574

(52) U.S. Cl. CPC C12Q 1/6886; C12Q 1/6869; G01N 33/57488; G01N 33/57407; C12Q 2600/156; C12Q 2600/158; C12Q 2600/106

(57) ABSTRACT

Provided herein are compositions, systems, kits, and methods for performing an activity based on detecting, in a sample from a cancer patient, the presence of elevated levels of Lactotransferrin (LTF) mRNA or protein, or detecting the presence in the MIF promoter region of at least one of: −173C and −794 CATT₅₋₈, and treating a patient with immunotherapy, or generating a report that the subject should be treated with immunotherapy.

The present application claims priority to U.S. Provisional application Ser. No. 63/112,461, filed Nov. 11, 2020, which is herein incorporated by reference in its entirety as if fully set forth herein.

### FIELD

Provided herein are compositions, systems, kits, and methods for performing an activity based on detecting, in a sample from a cancer patient, the presence of elevated levels of Lactotransferrin (LTF) mRNA or protein, or detecting the presence in the MIF promoter region of at least one of: −173C and −794 CATT₅₋₈, and treating a patient with immunotherapy, or generating a report that the subject should be treated with immunotherapy.

### BACKGROUND

Immunotherapeutic strategies to stimulate anti-cancer immune responses have provided new treatment options in multiple advanced cancers (1-4). However, the efficacy of these approaches is variable, and in some tumors, such as glioblastoma (GBM), immunotherapy success has been limited (5-7). The obstacles to immunotherapy effectiveness in GBM include a highly suppressive myeloid cell-driven tumor microenvironment and systemic immune suppression, which limits T cell infiltration and activation, and the anatomical limitations of the blood-brain and blood-tumor barriers (8-12). Accordingly, identifying how resistance to these therapies is regulated is essential for developing effective next-generation immunotherapeutic strategies for GBM and other refractory cancers.

Within the GBM microenvironment, a series of cell-cell interactions concomitantly drive tumor growth and immune suppression (10). An immune-suppressive pathway in GBM was identified that is driven by macrophage migration inhibitory factor (MIF) secreted by cancer stem cells (CSCs) that in turn activates myeloid-derived suppressor cells (MDSCs) (13). Recent has shown that MDSCs are increased in the circulation and tumor microenvironment (12), they portend a poor prognosis (8), their expansion can be driven by CSC-derived MIF (8,13), and they can be reduced by MIF neutralization (either genetically or pharmacologically) (9,14). Furthermore, MIF has been studied in a variety of cancers in the context of inflammation and has been found to regulate immune activity (15-32). However, MIF has not been explored in the context of immunotherapy.

### SUMMARY

Provided herein are compositions, systems, kits, and methods for performing an activity based on detecting, in a sample from a cancer patient, the presence of elevated levels of Lactotransferrin (LTF) mRNA or protein, or detecting the presence in the MIF promoter region of at least one of: −173C and −794 CATT₅₋₈, and treating a patient with immunotherapy, or generating a report that the subject should be treated with immunotherapy.

In some embodiments, provided herein are methods of performing an activity based on the presence of at least one polymorphism in the DNA of a patient with glioblastoma comprising: a) performing a nucleic acid detection assay on a DNA sample from a subject, or receiving results from the assay, wherein the assay detects the presence in the MIF promoter region of at least one polymorphism selected from: −173C and −794 CATT₅₋₈, and wherein the subject has symptoms of glioblastoma; and b) performing at least one of the following activities: i) treating the subject with: a glioblastoma therapeutic agent, an immune modulating therapy for glioblastoma, pembrolizumab, ipilimumab, nivolumab, a viral therapy for glioblastoma, or a CAR-T cell therapy for glioblastoma; ii) generating and/or transmitting a report that indicates the presence of the at least one polymorphism and that the subject has increased recurrence risk and/or more rapid decline in KPS status risk and/or decreased survival risk compared to glioblastoma patient's without one or both of the polymorphisms; and iii) generating and/or transmitting a report that indicates the presence of the at least one polymorphism, and that the subject should be treated with a glioblastoma therapeutic agent, an immune modulating therapy for glioblastoma, pembrolizumab, ipilimumab, nivolumab, a viral therapy for glioblastoma, or a CAR-T cell therapy for glioblastoma.

In certain embodiments, the subject's genotype is determined to be −173G/C or −173C/C. In further embodiments, the subject's genotype is determined to be −794 CATT₅-8/CATT₄ or CATT₅₋₈/CATT₅₋₈. In additional embodiments, the subject has both of the polymorphisms. In particular embodiments, the report indicates that the subject has both of the polymorphisms. In further embodiments, the −794 CATT₅₋₈ is −794 CATT₇. In other embodiments, the detecting is conducted by a method comprising sequencing (e.g., next generation sequencing, such as Illumina SBS technology).

In certain embodiments, step a) is receiving results from the assay, and wherein the at least one of the following activities is treating the subject. In additional embodiments, the treating is with the immune modulating therapy for glioblastoma. In other embodiments, the treating is with the CAR-T cell therapy for glioblastoma.

In some embodiments, provided herein are methods for performing an activity based on elevated levels of Lactotransferrin (LTF) in a biological sample from a patient with cancer comprising: a) performing a detection assay on a biological sample from a subject, or receiving results from the assay, wherein the assay detects an increased level of Lactotransferrin (LTF) mRNA and/or LTF protein compared to control levels, and wherein the subject has symptoms of cancer (e.g., a non-thyroid cancer); and b) performing at least one of the following activities: i) treating the subject with: an immune modulating therapy, pembrolizumab, ipilimumab, nivolumab, or a CAR-T cell therapy; ii) generating and/or transmitting a report that indicates the increased level of LTF mRNA and/or protein and that the subject should be treated with an immune modulating therapy, pembrolizumab, ipilimumab, nivolumab, or a CAR-T cell therapy.

In certain embodiments, the cancer is glioblastoma or melanoma. In particular embodiments, the LTF mRNA or LTF protein is the LTF mRNA. In some embodiments, the LTF mRNA or LTF protein is the LTF protein. In additional embodiments, step a) is receiving results from the assay, and wherein the at least one of the following activities is treating the subject. In certain embodiments, the treating is with the immune modulating therapy. In other embodiments, the immune modulating therapy is immune modulating therapy for glioblastoma or melanoma. In additional embodiments, the treating is with the CAR-T cell therapy. In other embodiments, the CAR-T cell therapy is CAR-T cell therapy for glioblastoma or melanoma.

### DESCRIPTION OF THE FIGURES

FIG. 1. Patients with the MIF SNP rs755622 have an increase in Lactotransferrin (LTF) and increased immune microenvironment signaling. A) Expression of the MIF gene mRNA in TCGA_GBMLGG was compared among the histology subtypes astrocytoma, oligodendroglioma, oligoastrocytoma, and GBM using unpaired t-test. B) The MIF gene structure highlighting the −794 CATT repeat, which contains between 5 and 8 repeats, and the MIF SNP rs755622 at position −173. C) Using GBM samples from n=17 G/G genotype patients and n=17 C/G patients, differential expression analysis was performed using DESeq2 on raw counts and the log fold change genes>1 and with p value>1−log 10(adjusted p-value). D) The 25 genes with both the largest increases and largest decreases in gene expression by fold change are shown via heatmap, and rows are clustered using hierarchical clustering; transcripts per million are scaled to row for heatmap color scale. E) GSEA (gene set enrichment analysis) was performed using Hallmark gene sets on the differentially expressed gene list comparing the C/G genotype to the G/G genotype sorted by log 2 fold change for gene rank position. NES score is shown in red/blue with red enriched in C/G and blue enriched in G/G, while the size of the circle represents the −log 10 p value as determined by GSEA. ssGSEA analysis was performed using the R package GSVA for cell type gene signatures to deconvolute immune cell types, and microenvironment, stromal, and immune scores were generated using xCell deconvolution R package from the bulk RNA-seq available for each of the n=34 patients. ANOVA analysis was performed to compare the deconvolution scores between C/G and G/G patients, with the p value shown above the heatmap of the scaled scores. G) ssGSEA scores for significant populations are shown as a boxplot with unpaired t-test p values shown for each.

FIG. 2. Immunofluorescence confirms enhanced T cell infiltration and CD8 T cell activation in GBM patients with the MIF SNP rs755622. A) LTF expression was analyzed by immunofluorescence for n=22 samples matched from the RNAseq analysis to compare patients with the minor allele (C/G) and those with the major allele (G/G), with representative images of the dataset shown as a whole view of the slide in the heatmap images (left column, yellow=increased density of staining). In a 20× image of the slide, cells are outlined in white, with nuclei marked by DAPI and LTF pseudocolored purple. B) The average LTF expression (mean fluorescence intensity) of cells per sample was compared between each genotype and the negative secondary-only control. C) The quantity of LTF-positive cells per total area was measured and then compared between the C/G and G/G genotypes using unpaired t-test. D) The percent of LTF-positive cells of the total cells per sample was compared between each genotype group and further subdivided into prognostic categories based on greater than or less than median overall survival. E) Staining for CD3 and CD8 markers to determine CD8+ T cell infiltration, with representative images of each genotype and prognosis group (CD3=yellow, and CD8=red, DAPI=blue). Percent total T cells F) and CD8+ T cells G) of total cells per sample comparing the genotype/prognosis categories only. H) CD107a expression of CD8+ T cells from all samples was compared between prognoses. I) CD107a expression of CD8+ T cells was compared between the C/G genotype and G/G genotype.

FIG. 3. The MIF SNP rs755622 correlates with reduced macrophages and increased T cell infiltration by immunofluorescence. A) A multiplex myeloid panel of antibodies was developed and included HLA-DR, CD68, CD74, CD11b, P2Ry12, and CD4, with the average expression shown for each cell type identified. B) Representative images of each marker and pseudocoloring of each final cell type is shown in one representative image containing all cell types. C) Analysis of the percent of macrophages, MDSCs, ratio of MDSC/CD8+ T cells, microglia, monocytes, and CD4+ T cells was performed using the percent of each cell type per sample out of total cells identified. D) Correlation analyses were performed for each patient's immune cell infiltrates from major allele genotype (G/G) patients using LTF staining, myeloid staining, and lymphocyte staining. Color scale and circle size are representative of the Pearson correlation coefficient. E) Correlation analysis of staining cohorts for only the minor allele patients (C/*).

FIG. 4. GBM patients with high LTF expression are immunologically activated. A) TCGA_GBM mRNA seq data was analyzed to compare LTF-high (top 25% expression) and LTF-low (bottom 25% expression) patients, and differentially expressed genes are show between the groups. B) GSEA analysis was performed based on the ranked gene list from differential expression between LTF-high and LTF-low samples, with pathways enriched in LTF-high samples shown in red. C) Highlighted GSEA plots of rank ordered genes from the Hallmark pathways for allograph rejection, complement, which are enriched in LTF-high patients, and Mitotic Spindle and Myc Targets pathways enriched in the LTF-low patients. D) Deconvolution analysis of the samples belonging the LTF-high and LTF-low groups showed increased immune cell type infiltration and increased immune scores, with ANOVA analysis for multiple comparisons showing the p-value by heatmap coloring. E). Individual plots for significantly different estimated cell types and scores shown as individual boxplots with unpaired t-test. F) GBM clinical trial of nivolumab+bevacizumab (NCT03452579) with clinical outcomes as partial, progression, and stable were genotyped for the MIF SNP rs755622 and compared.

FIG. 5. LTF expression is associated with immunotherapy response. A) ssGSEA score for LTF was used to identify the top 25% of samples with the highest expression levels in each of four immunotherapy clinical trials of melanoma. B-F) Univariant survival analyses were performed for each cohort individually and then for all cohorts together, with log-rank p value shown. G) TIDE biomarker analysis was utilized via the TIDE website using LTF as a single gene biomarker and identified 9 datasets where AUC>0.5. H) Deconvolution analysis of melanoma datasets showing top 25% (highest expression) vs bottom 75% (lowest expression) of samples shows statistically significant changes in scores analyzed by ANOVA. I) Individual analysis of immune score, microenvironment score, and stromal score along with increased T cells, IFNG and cytotoxic cells. J) Response analysis was performed comparing the high- and low-LTF groups by previously defined Recist response categories, and Fisher's exact test was performed to compare the two groups, with the p value=0.055.

FIG. 6. GTEX database analysis of MIF and rs755622 as expression quantitative loci across tissues. Searching the GTEX database for rs755622 and its association across multiple tissues for a link with MIF expression shows significance in many tissues except central nervous system related tissues.

FIG. 7. Univariate and multivariable analysis of the MIF SNP rs755622 in GBM cohorts. Germline DNA was acquired from PBMCs or saliva samples from n=449 (Cleveland Clinic), n=386 (Moffitt Cancer Center), and n=131 (Case Western) GBM patients and then tested for the MIF SNP rs755622 using PCR with restriction enzyme digestion as described in detail in the methods. A) Descriptive statistics of clinical features of GBM including sex, surgery type, standard of care, KPS, and SNP status including all cohorts of GBM patients. Primary features known to associate with outcome such as higher KPS, total as compared to subtotal tumor resection and treatment with standard of care all confer a survival advantage. B) Descriptive statistics combining data from all three cohorts of GBM patients demonstrated statistically significant differences in allele frequencies of the MIF SNP rs755622 (minor ‘C’ allele containing genotypes vs the homozygous major ‘G’ allele genotype) for standard of care treatment and sex status. C) Allele frequencies in MIF rs755622 are similar in the 3 GBM cohorts and the 1000 genomes European cohort using Hardy-Weinberg principle and chi-square test for differences from reference control cohort. D) Univariate analysis of overall survival across all cohorts shows no survival difference by log rank test. E). Restricting to patients uniformly treated with standard of care, we observed no significant difference in overall survival according to MIF rs755622 genotype (CC/CG versus GG) by the log rank test. F). Univariate and multivariable Cox proportional hazards models predicting overall and progression-free survival for MIF SNP rs755622 in GBM cohorts.

FIG. 8. MIF CATT repeat analysis in GBM patients shows correlation with rs755622. A) The CATT microsatellite in the MIF promoter upstream of the MIF SNP rs755622 was analyzed by capillary electrophoresis in the Cleveland Clinic cohort. C) Those patients with both rs755622 SNP information and CATT repeat information were correlated to confirm linkage disequilibrium, which showed approximately 95% segregation of the 5/6 repeat and the major allele at rs755622, while 7/8 CATT repeats were linked with the minor allele of rs755622.

FIG. 9. Univariate analysis of clinical cohorts for MIF snp rs755622 (Overall Survival). A-F) Univariate analysis of overall survival comparing patients with the minor allele to patients homozygous for the major allele across all samples (left column) and then those who received full standard of care treatment protocol (right column), with log rank p-value comparing each group provided to the right of each curve.

FIG. 10. Univariate analysis of clinical cohorts for MIF snp rs755622 (Progression Free Survival). A-F) Univariate analysis of recurrence-free survival comparing patients with the minor allele to patients homozygous for the major allele across all samples (left column) and then those who received full standard of care treatment protocol (right column), with log rank p value comparing each group provided to the right of each curve.

FIG. 11. Ingenuity pathway analysis of C/G (rs755622) vs homozygous dominant. Genes from the differential expression analysis of rs755622 minor allele patients vs major allele patients were ranked by log fold change and the top100 genes increased in minor allele and top 100 decreased in minor allele were utilized for Ingenuity pathway analysis. (A-B) Pathway description and p-value are shown for the top 25 pathways associated with the 100 genes.

FIG. 12. TCGA_GBM analysis of the MIF SNP rs2096525 identifies increased immune signaling. A) Analysis of TCGA_GBM whole exome sequencing for rs2096525 identified 12.2% of patients with the minor allele. B) The TCGA_GBM mRNA sequencing data and genotype information obtained from the whole exome sequencing data (n=159) were used for differential expression analysis between patients with the minor allele and patients homozygous for the major allele. Volcano plot represents deseq2 analysis of differential expression between the patients with the rs2096525 minor allele and patients homozygous for the major allele. C) GSEA analysis of Hallmark pathways from differential expression analysis of rs2096525 patients demonstrates increased pathways in SNP-containing patients. D) Selected enriched top two pathways are shown for GSEA analysis, with ranked genes in each pathway for enrichment and the top two downregulated pathways also shown. E) Deconvolution analysis using ssGSEA with cell type gene sets from Nanostring demonstrates increased macrophages, neutrophils, and T cell populations in samples from patients with the minor allele rs2096525 SNP.

### DETAILED DESCRIPTION

Provided herein are compositions, systems, kits, and methods for performing an activity based on detecting, in a sample from a cancer patient, the presence of elevated levels of Lactotransferrin (LTF) mRNA or protein, or detecting the presence in the MIF promoter region of at least one of: −173C and −794 CATT₅₋₈, and treating a patient with immunotherapy, or generating a report that the subject should be treated with immunotherapy.

In certain embodiments, the present disclosure relates to detecting a migration inhibitory factor (MIF) −794 CATT repeat and/or MIF rs755622 single nucleotide polymorphism (SNP) as prognostic factors and response prediction in immunotherapy treatment in glioblastoma (GBM), where rs755622=MIF-173G/C rs5844572=−794CATT₅-8.

In work conducted during the development of embodiments herein, a ‘The Cancer Genome Atlas’ (TCGA) data screen was performed on GBM patients to evaluate the promoter region of MIF. Subsequently, peripheral nucleated blood from 200 GBM patients was obtained and analyzed for the two unique MIF markers. MIF promoter variations in patients were compared to frequencies in the general population. The TCGA data screen indicated that younger GBM patients harbor the variant MIF SNP. In an initial study demonstrating feasibility of the screen and MIF nucleotide variability, it was found that patients with GBM tended to have increased MIF CATT repeats when compared to proportions in the general population, and the repeat numbers were approaching levels seen in other diseased immunogenic populations. Within GBM subsets, patients with increased CATT repeats had earlier time to recurrence, more rapid declines in Kamofsky Performance Status (KPS), and decreased survival. Individuals who have variant MIF microsatellite loci develop GBM at an earlier age, and harbor more aggressive tumors. Analysis of MIF promoter sequences in the peripheral blood may be used as a screening tool for both the development of GBM and as a marker for increased aggressiveness and/or response to immune therapies in patients with glioblastoma.

In work conducted during the development of embodiments herein, it was found that Intra-tumoral MIF expression leads to a worse prognosis in GBM. Genomic presence of minor allele SNPs confers a 3.1 month decrease in progression free survival and a 4.1 month decrease in overall survival in GBM despite standard of care therapies. Using a multivariate analysis, minor allele SNP presence is an independent prognostic indicator for worse outcomes in GBM Minor allele SNP presence is more prognostic than KPS, MGMT status, sex, 1p19q co-deletion MDSC signature genes are overexpressed in patients with genomic presence of minor allele SNPs

The present disclosure is not limited with regard to how the MIF promoter regions variants (−173C and −794 CATT₅₋₈) are detected. In some embodiments, detection involves measurement or detection of a characteristic of a non-amplified nucleic acid, amplified nucleic acid, a component comprising amplified nucleic acid, or a byproduct of the amplification process, such as a physical, chemical, luminescence, or electrical aspect, which correlates with amplification (e.g. fluorescence, pH change, heat change, etc.). In some embodiments, fluorescence detection methods are provided for detection of amplified or non-amplified MIF promoter region nucleic acid. In certain embodiments, various detection reagents, such as fluorescent and non-fluorescent dyes and probes are employed. For example, the protocols may employ reagents suitable for use in a TaqMan reaction, such as a TaqMan probe; reagents suitable for use in a SYBR Green fluorescence detection; reagents suitable for use in a molecular beacon reaction, such as molecular beacon probes; reagents suitable for use in a scorpion reaction, such as a scorpion probe; reagents suitable for use in a fluorescent DNA-binding dye-type reaction, such as a fluorescent probe; and/or reagents for use in a LightUp protocol, such as a LightUp probe. In some embodiments, provided herein are methods and compositions for detecting and/or quantifying a detectable signal (e.g. fluorescence) from MIF promoter region target nucleic acid. Thus, for example, methods may employ labeling (e.g. during amplification, post-amplification) amplified nucleic acids with a detectable label, exposing partitions to a light source at a wavelength selected to cause the detectable label to fluoresce, and detecting and/or measuring the resulting fluorescence. Fluorescence emitted from label can be tracked during amplification reaction to permit monitoring of the reaction (e.g., using a SYBR Green-type compound), or fluorescence can be measure post-amplification.

In some embodiments, detection of MIF promoter regions variants (−173C and −794 CATT₅-8) employs one or more of fluorescent labeling, fluorescent intercalation dyes, FRET-based detection methods (U.S. Pat. No. 5,945,283; PCT Publication WO 97/22719; both of which are incorporated by reference in their entireties), quantitative PCR, real-time fluorogenic methods (U.S. Pat. No. 5,210,015 to Gelfand, U.S. Pat. No. 5,538,848 to Livak, et al., and U.S. Pat. No. 5,863,736 to Haaland, as well as Heid, C. A., et al., Genome Research, 6:986-994 (1996); Gibson, U. E. M, et al., Genome Research 6:995-1001 (1996); Holland, P. M., et al., Proc. Natl. Acad. Sci. USA 88:7276-7280, (1991); and Livak, K. J., et al., PCR Methods and Applications 357-362 (1995), each of which is incorporated by reference in its entirety), molecular beacons (Piatek, A. S., et al., Nat. Biotechnol. 16:359-63 (1998); Tyagi, S. and Kramer, F. R., Nature Biotechnology 14:303-308 (1996); and Tyagi, S. et al., Nat. Biotechnol. 16:49-53 (1998); herein incorporated by reference in their entireties), Invader assays (Third Wave Technologies, (Madison, Wis.)) (Neri, B. P., et al., Advances in Nucleic Acid and Protein Analysis 3826:117-125, 2000; herein incorporated by reference in its entirety), nucleic acid sequence-based amplification (NASBA; (See, e.g., Compton, J. Nucleic Acid Sequence-based Amplification, Nature 350: 91-91, 1991; herein incorporated by reference in its entirety), Scorpion probes (Thelwell, et al. Nucleic Acids Research, 28:3752-3761, 2000; herein incorporated by reference in its entirety), partially double-stranded linear probes (Luk, K.-C., et al, J. Virological Methods 144:1-11, 2007; herein incorporated by reference in its entirety), capacitive DNA detection (See, e.g., Sohn, et al. (2000) Proc. Natl. Acad. Sci. U.S.A. 97:10687-10690; herein incorporated by reference in its entirety), etc.

Target MIF promoter nucleic acid molecules may be analyzed by any number of techniques to determine the presence of, amount of, or identity of the molecule. Non-limiting examples include sequencing, mass determination, and base composition determination. The analysis may identify the sequence of all or a part of the amplified nucleic acid (e.g., MIP promoter region containing position −173C and −794) or one or more of its properties or characteristics to reveal the desired information.

Illustrative non-limiting examples of nucleic acid sequencing techniques include, but are not limited to, chain terminator (Sanger) sequencing and dye terminator sequencing, as well as “next generation” sequencing techniques. A number of DNA sequencing techniques are known in the art, including fluorescence-based sequencing methodologies (See, e.g., Birren et al., Genome Analysis: Analyzing DNA, 1, Cold Spring Harbor, N.Y.; herein incorporated by reference in its entirety). In some embodiments, automated sequencing techniques understood in that art are utilized. In some embodiments, the systems, devices, and methods employ parallel sequencing of partitioned amplicons (PCT Publication No: WO2006084132 to Kevin McKeman et al., herein incorporated by reference in its entirety). In some embodiments, DNA sequencing is achieved by parallel oligonucleotide extension (See, e.g., U.S. Pat. No. 5,750,341 to Macevicz et al., and U.S. Pat. No. 6,306,597 to Macevicz et al., both of which are herein incorporated by reference in their entireties). Additional examples of sequencing techniques include the Church polony technology (Mitra et al., 2003, Analytical Biochemistry 320, 55-65; Shendure et al., 2005 Science 309, 1728-1732; U.S. Pat. Nos. 6,432,360, 6,485,944, 6,511,803; herein incorporated by reference in their entireties) the 454 picotiter pyrosequencing technology (Margulies et al., 2005 Nature 437, 376-380; US 20050130173; herein incorporated by reference in their entireties), the Solexa single base addition technology (Bennett et al., 2005, Pharmacogenomics, 6, 373-382; U.S. Pat. Nos. 6,787,308; 6,833,246; herein incorporated by reference in their entireties), Illumina Single base sequencing technology, the Lynx massively parallel signature sequencing technology (Brenner et al. (2000). Nat. Biotechnol. 18:630-634; U.S. Pat. Nos. 5,695,934; 5,714,330; herein incorporated by reference in their entireties) and the Adessi PCR colony technology (Adessi et al. (2000). Nucleic Acid Res. 28, E87; WO 00018957; herein incorporated by reference in its entirety).

In certain embodiments, the LTF mRNA is detected by Northern blot analysis, nuclease protection assays (NPA), in situ hybridization, reverse transcription-polymerase chain reaction (RT-PCR), or RNA-seq sequencing methods.

In particular embodiments, the Lactotransferrin (LTF) protein is detected by a protein detection assay. In some embodiments, the LTF protein detection assays include, but are not limited to: 1) a sandwich immunoassay (e.g., monoclonal, polyclonal and/or DVD-Ig sandwich immunoassays or any variation thereof (e.g., monoclonal/DVD-Ig or DVD-Ig/polyclonal), including chemiluminescence detection, radioisotope detection (e.g., radioimmunoassay (RIA)) and enzyme detection (e.g., enzyme immunoassay (EIA) or enzyme-linked immunosorbent assay (ELISA) (e.g., Quantikine ELISA assays, R&D Systems, Minneapolis, Minn.))), 2) a competitive inhibition immunoassay (e.g., forward and reverse), 3) a fluorescence polarization immunoassay (FPIA), 4) an enzyme multiplied immunoassay technique (EMIT), 5) a bioluminescence resonance energy transfer (BRET), 6) a homogeneous chemiluminescent assay, 7) a SELDI-based immunoassay, 8) chemiluminescent microparticle immunoassay (CMIA) and 9) a clinical chemistry colorimetric assay (e.g., IMA, creatinine for eGFR determination and LC-MS/MS). (See, e.g., Tietz Textbook of Clinical Chemistry and Molecular Diagnostics. 4th Edition, edited by C A Burtis, E R Ashwood and D E Bruns, Elsevier Saunders, St. Louis, Mo., 2006).

Further, if an immunoassay is being utilized, any suitable detectable label as is known in the art can be used. For example, the detectable label can be a radioactive label (such as 3H, 1251, 35S, 14C, 32P, and 33P), an enzymatic label (such as horseradish peroxidase, alkaline peroxidase, glucose 6-phosphate dehydrogenase, and the like), a chemiluminescent label (such as acridinium esters, thioesters, or sulfonamides; luminol, isoluminol, phenanthridinium esters, and the like), a fluorescent label (such as fluorescein (e.g., 5-fluorescein, 6-carboxyfluorescein, 3′6-carboxyfluorescein, 5(6)-carboxyfluorescein, 6-hexachloro-fluorescein, 6-tetrachlorofluorescein, fluorescein isothiocyanate, and the like)), rhodamine, phycobiliproteins, R-phycoerythrin, quantum dots (e.g., zinc sulfide-capped cadmium selenide), a thermometric label, or an immuno-polymerase chain reaction label. An introduction to labels, labeling procedures and detection of labels is found in Polak and Van Noorden, Introduction to Immunocytochemistry, 2nd ed., Springer Verlag, N.Y. (1997), and in Haugland, Handbook of Fluorescent Probes and Research Chemicals (1996), which is a combined handbook and catalogue published by Molecular Probes, Inc. Eugene, Oreg. A fluorescent label can be used in FPIA (see, e.g., U.S. Pat. Nos. 5,593,896, 5,573,904, 5,496,925, 5,359,093, and 5,352,803, which are hereby incorporated by reference in their entireties). An acridinium compound can be used as a detectable label in a homogeneous or heterogeneous chemiluminescent assay (see, e.g., Adamczyk et al., Bioorg. Med. Chem. Lett. 16: 1324-1328 (2006); Adamczyk et al., Bioorg. Med. Chem. Lett. 4: 2313-2317 (2004); Adamczyk et al., Biorg. Med. Chem. Lett. 14: 3917-3921 (2004); and Adamczyk et al., Org. Lett. 5: 3779-3782 (2003)).

### EXAMPLES

### Example 1

Identification of a MIF SNPs, and Lactotransferrin, that Alters the Immune Landscape in the Tumor Microenvironment and Informs Immunotherapy Response

While immunotherapies have shown durable responses for multiple tumors, their efficacy remains limited in some advanced cancers, including glioblastoma. This may be due to differences in the immune landscape, as the glioblastoma microenvironment strongly favors immunosuppressive myeloid cells, which are linked to an elevation in immune-suppressive cytokines, including macrophage migration inhibitory factor (MIF). We now find that a single-nucleotide polymorphism (SNP) rs755622 in the MIF promoter associates with increased leukocyte infiltration in glioblastoma and can be leveraged to predict immunotherapy response across multiple cancers. Furthermore, we identified lactotransferrin expression as being associated with the SNP, which could also be used as a biomarker for immune infiltrated tumors with a higher response rate to immunotherapy. These findings provide the first example in glioblastoma of a germline SNP that underlies differences in the immune microenvironment and identifies high lactotransferrin expression as a biomarker of immunotherapy response.

### Materials and Methods

### Subjects and Methods

For Cleveland Clinic, peripheral blood samples from 451 patients with GBM were collected through the Rose Ella Burkhardt Brain Tumor and Neuro-Oncology Center under approved IRB protocol 2559. White blood cells from each blood sample were isolated via Ficoll gradient and then snap frozen and stored at −80° C. for research use. For this study, we selected all available GBM samples. For Moffitt Cancer Center, salivary DNA samples collected using Origene kits was available for 386 recently diagnosed GBM patients under IRB protocol MCC 15004. DNA was extracted and stored in aliquot pellets at −80° C. for future research use. For Case Western Reserve University (CWRU)/University Hospitals of Cleveland, peripheral blood samples from 131 patients with GBM were collected through the Ohio Brain Tumor Study (OBTS) at Case Western Reserve University (CWRU), under approval from University Hospitals IRB CC296. Clinical and pathological data were gathered for each patient. Patient blood samples were collected and processed at the time of consent.

### DNA Isolation and Quantification

Genomic DNA was extracted from the peripheral blood of GBM patients using a Qiagen DNeasy Blood & Tissue Kit following the manufacturer's protocols. DNA purity and concentration were measured using a ThermoFisher NanoDrop spectrophotometer.

### SNP Genotyping

MIF SNP genotyping was performed using PCR amplification and subsequent restriction enzyme digestion with AluI. PCR was performed using Accuprime Pfx DNA polymerase (ThermoFisher, catalog number 12344-024) using 0.2 μmol forward primer (5′-CCCAAAGACAGGAGGTAC-3′, SEQ ID NO:1) and 0.2 μmol reverse primer (5′-ATGATGGCAGAAGGACCAG-3′, SEQ ID NO:2). PCR was run as follows: 95° C. for 5 minutes, followed by 35 cycles of 95° C. for 30 seconds, 60° C. for 45 seconds, and 68° C. for 1 minute. Following the 35 cycles, there was a final 68° C. elongation step for 5 minutes, followed by storage at 4° C. After amplification, the PCR product was confirmed on a 1% agarose gel by identification of an approximately 500 bp product. After confirmation, 10 μl of PCR product was mixed with 2 μl 10× CutSmart buffer. 1 μl AluI, and 7 μl water and digested at 37° C. for 1 hour. After digestion, the alleles containing a G (non SNP) produced a 450 bp fragment, while the alleles with a C (rs755622) produced a 270 bp fragment.

### SNP Calling

Raw BAM files from the TCGA_GBM cohort were utilized to analyze the rs2096525 MIF SNP from whole exome sequencing data aligned by the TCGA. Aligned the SNP rs2096525 genotype was identified via use of HaplotypeCaller, where samples with alternative counts at the reference position chr22: 23894632 were identified. After classification of the samples by genotype, the phenotype data was downloaded via TCGA, and survival analysis was performed using log-rank test via R version 4.1.0.

### RNA Sequencing

Flash-frozen tissue was requested from the Rose Ella Burkhardt Brain Tumor Center at the Cleveland Clinic under IRB 2559 and corresponded to n=34 patients with previously identified rs755622 SNP status from matched white blood cell pellets (n=17 C/*, and n=17 G/G). Within each group, patients were selected who underwent full Stupp protocol standard-of-care treatment and were evenly divided by sex and prognosis (<6 months progression-free survival (PFS) or >6 months PFS). Samples were processed and sequenced by Genewiz. Briefly, RNA was extracted by Qiagen RNeasy kit, and then the library was prepared using True-seq library preparation. Average sequencing depth was 40 Mbp per sample.

FASTQ files were aligned to the hg19 using STAR aligner with default parameters. Fragments were counted using Rsamtools with UCSC.hg19.knownGene transcript database. Raw counts were used in DESeq2 downstream for differential expression comparing the rs75662 SNP status groups.

### TCGA RNAseq Data

Processed count data from TCGA_GBM mRNA dataset was downloaded from the Broad Firehose “http://gdac_broadinstitute.org/runs/stddata_2016_01_28/data/GBM/20160128/”, where the raw count file GBM.uncv2.mRNAseq_raw_counts.txt was utilized for downstream analysis.

### Differential Expression

Raw counts from TCGA_GBM were analyzed using the R package DESeq2 version 1.29 in R version 4.0.1. After identification of the germline SNP status of rs2096525, the patient samples containing the minor allele were compared to patients homozygous for the major allele for differential expression.

ssGSEA

The nCounter PanCancer Immune Profiling Panel gene set was used for immune infiltration deconvolution signatures. Each signature was used with ssGSEA using Gene Set Variation Analysis (GSVA) R package version 1.40.1³⁴. Microenvironment score, stromal score and immune score were generated using xCell R package version 1.1.0³⁵. Comparing signature scores between groups was performed using ANOVA with the p values shown for each comparison in the heatmaps above deconvolution heatmaps. All heatmaps of deconvolution and p-values were generated using pheatmap version 1.0.12.

### TIDE Evaluation of Biomarkers

Tumor Immune Dysfunction and Exclusion biomarker evaluation was performed at http://tide.dfci.harvard.edu/. Using LTF as a single-gene signature, the AUC for 25 immunotherapy studies was performed from RNA-sequencing data evaluating clinical response. Of the 25 signatures, 9 had an AUC greater than random³⁶.

### Melanoma Dataset Mining

Melanoma RNAseq datasets were utilized from published reports³⁷⁻⁴⁰ sGSEA was utilized to score the LTF gene in all samples, and then a top quartile cutoff was selected to group the samples into high and low groups. Univariate analysis was performed using the LTF score, and log rank p-values were obtained along with survival curves using the R package Survival.

### Immunofluorescence Staining

Serial sections (7 μm thick) from each sample (formalin-fixed paraffin-embedded tumor biopsies) were stained with 3 different sets of markers and indicated below:

Set 1 (triple immunofluorescence staining): DAPI, CD3 (ab11089, abcam, 1:50), CD8 (85336, cell signaling, 1:50), CD107a (NBP2-52721, novus, 1:50)
Set 2 (double immunofluorescence staining): DAPI, MIF (MAB2892, R&D systems, 1:500), LTF (HPA059976, Atlas, 1:100)
Set 3 (multiplex staining): DAPI, CD74 (ab1794,Abcam/1:200), CD11b (ab133357,Abcam/1:200 P2RY12 (NBP2-33870,Novus Bio/1:200), HLA-DR (ab20181/Abcam/1:200), CD68 (790-2931, Ventana/1:200), CD4 (ab133616, Abcam/1:200)

For staining, slides were baked at 60^{C} prior to deparrifinization. Slides were then deparaffinized using a Leica Autostaine XL and antigen retrieval was performed using a sodium citrate buffer (pH6) with slides steamed in pressure cooker at 110^{C} for 10 minutes. Slides were then cooled to room temperature and transferred to water for 5 minutes prior to TBST for 15 minutes. Primary antibody placed at above concentrations and incubated in a humid chamber overnight at 4^{C}. Slides placed on Biocare Intellipath Staining platform for blocking (3% donkey serum). secondary antibody incubations and Hoechst/DAPI staining (Thermo Scientific H1399/1 mg/ml). The following secondary antibodies were used: Rabbit Cy3 (Jackson Immuno 711-165-152, 1:250), Mouse 488 (Jackson Immuno 715-545-151, 1:250), Rat Cy3 (Jackson Immuno 712-165-153, 1:250), Rabbit Cy5 (Jackson Immuno 711-175-152, 1:250). Slides manually cover slipped with an aqueous mounting medium.

### Imaging

Whole tissue sections were imaged with multispectral capabilities of the Vectra Polaris Automated Quantitative Pathology Imaging System (Akoya Bioscience Inc.). Multispectral images were then unmixed in inForm (Akoya Biosciences Inc., version 2.5) to obtain component images for each individual marker and tissue autofluorescence. Component image tiles were stitched and saved as OME-TIFF (Open Microscopy Environment) format for analysis, storage, and archival.

### Image Analysis

The open source image analysis software QuPath⁴¹ was used for the detection and classification of cells. For each slide, 10 to 20 regions of interest (ROI) were selected to represent the different parts of the whole section while avoiding imaging, staining and sectioning artifacts. StarDist⁴², a deep-learning algorithm, along with a pretrained model was used within QuPath for detecting cell nuclei from the DAPI channel. For each cell, intensity measurements were used to determine its positivity for each marker in the panel. A custom script with a manual decision tree⁴³ was implemented in QuPath to classify cells based on their positivity. Representative images were extracted using QuPath, and single-cell data for each sample were exported as .csv files for further analysis and charting in R version 4.0.1.

### Descriptive Statistics and Survival Analysis

Demographic and clinical characteristics were evaluated between clinical cohorts. Analysis of variance (ANOVA) and chi squared tests were performed to assess differences in continuous and categorical variables, respectively. Additionally, these characteristics were assessed for MIF SNP rs755622. For this assessment, T tests were performed to assess differences in continuous data. All statistics were generated in R version 4.0.1.

### Survival Analysis

Overall and progression-free survival of each clinical cohort was assessed for MIF snp rs755622. Kaplan Meier (KM) analysis was performed to evaluate the difference in survival and recurrence between GG genotype patients and CC or CG genotype patients. These analyses were also performed among only those cases who had received standard of care. Log rank tests were performed to assess differences in KM curves. Univariate and multivariable Cox proportional hazards models were generated to assess the impact of MIF snp rs755622 on overall and progression free survival. The proportional hazards assumption was assessed and not found in violation. Multivariable models were adjusted for age, sex, surgery, and standard of care. Hazard ratios (HR) and 95% confidence intervals (95% CI) are reported. All statistics were generated in R version 4.0.1.

### Results

Patients with the MIF SNP Rs755622 have an Increase in Lactotransferrin (LTF) and Increased Immune Microenvironment Signaling

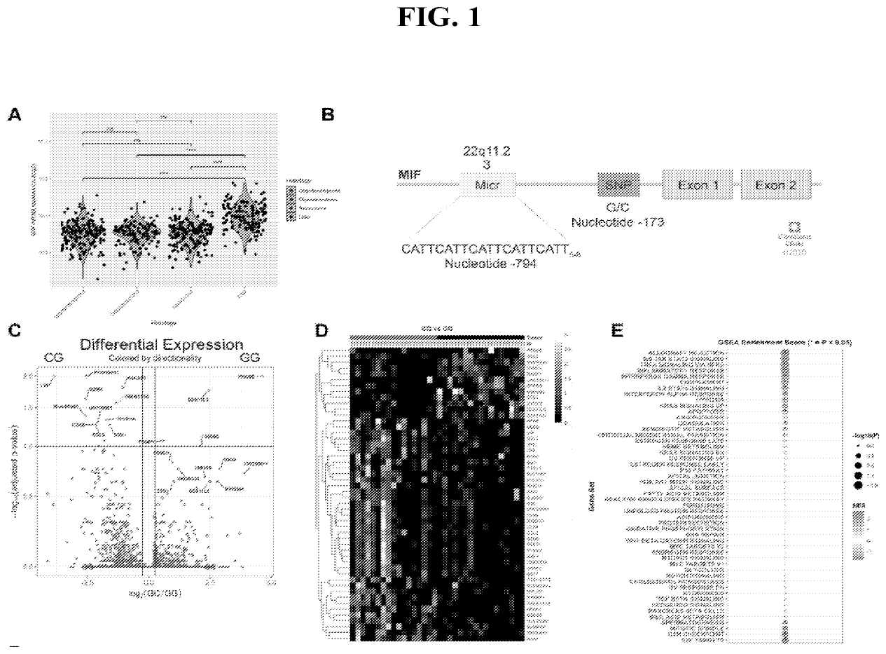

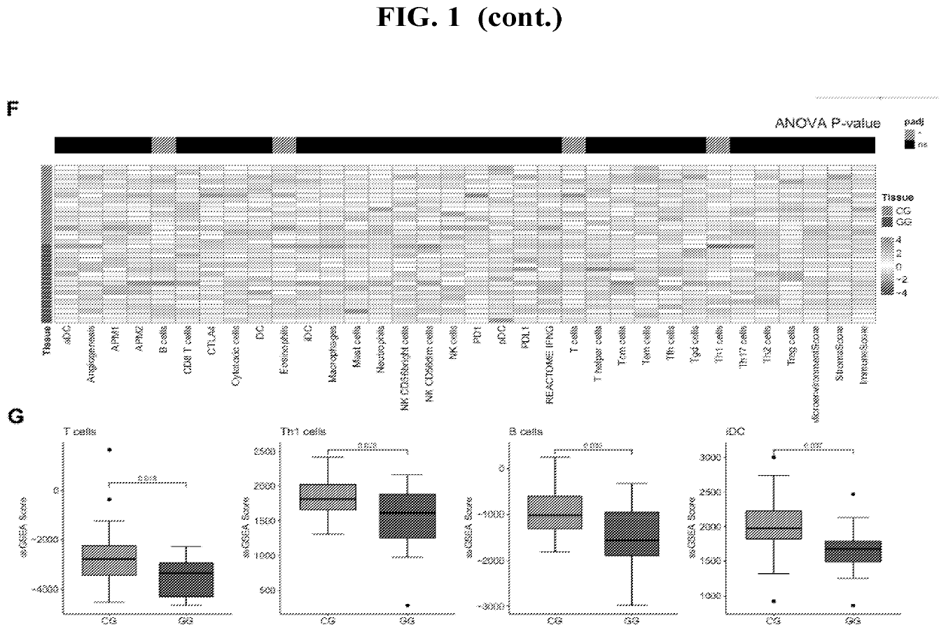

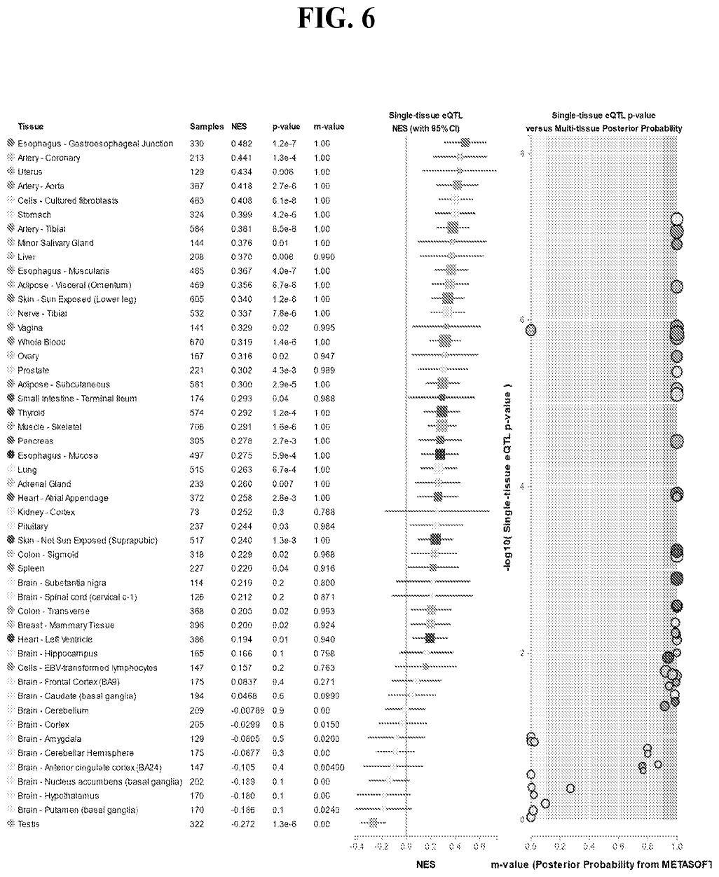

Based on our previous assessments of MIF as a driver of CSC-MDSC-mediated communication¹³, we assessed overall MIF expression levels across brain tumors and found elevated MIF in isocitrate dehydrogenase (IDH) wild-type GBM patient tumor samples when compared to that of patients with lower-grade (IDH mutant astrocytomas and oligodendrogliomas) gliomas (FIG. 1A). Furthermore, the Genotype-Tissue Expression (GTEx) project identifies the MIF snp rs755622 as an expression quantitative trait loci highly associated with expression in most tissues except the central nervous system, where it has almost opposing results (FIG. 6A and FIG. 6B). Based on these observations we hypothesized that GBM patients may have an increased prevalence of the regulatory MIF rs755622 nucleotide −173 G/C SNP (FIG. 1B).

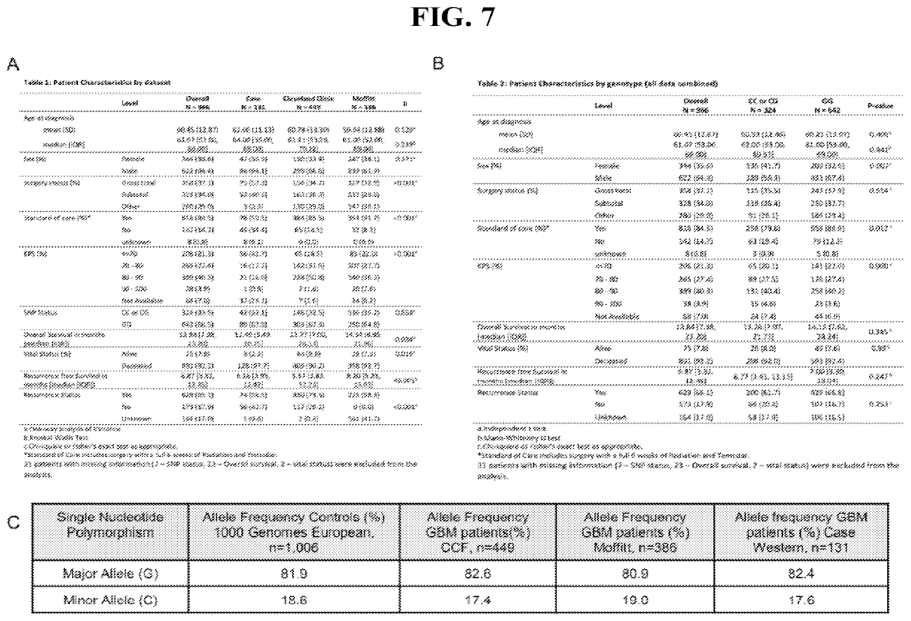

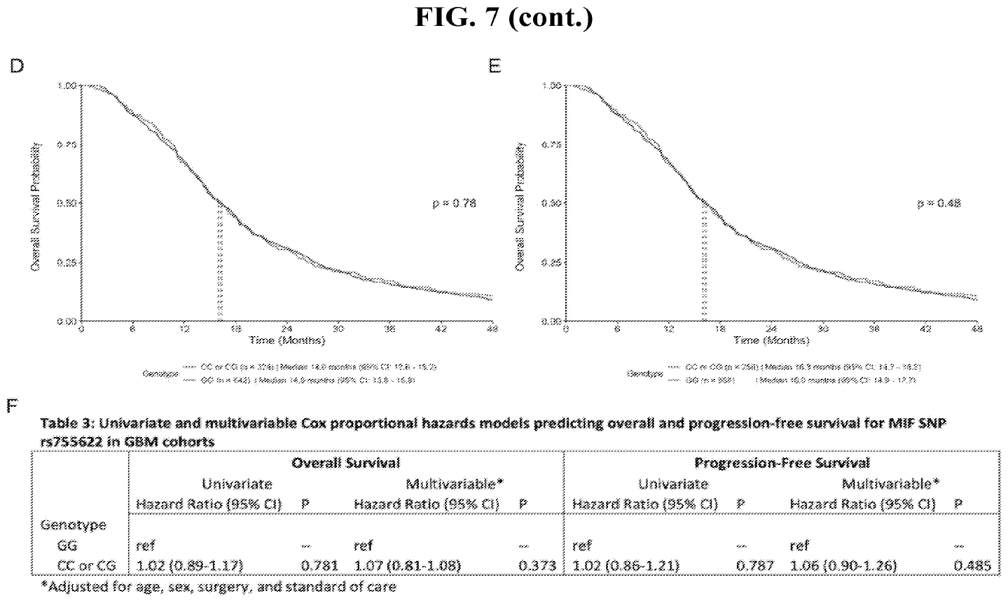

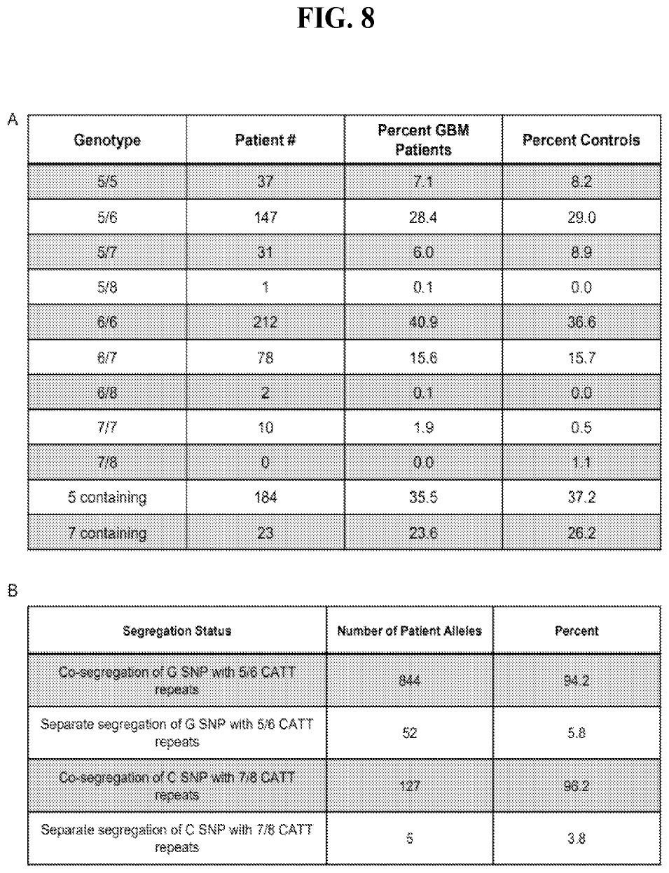

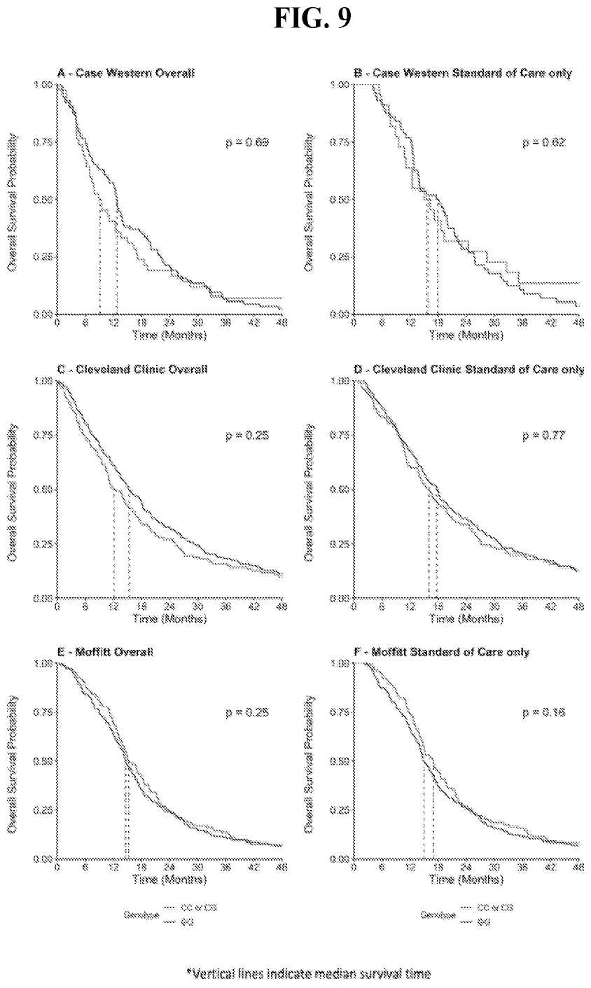

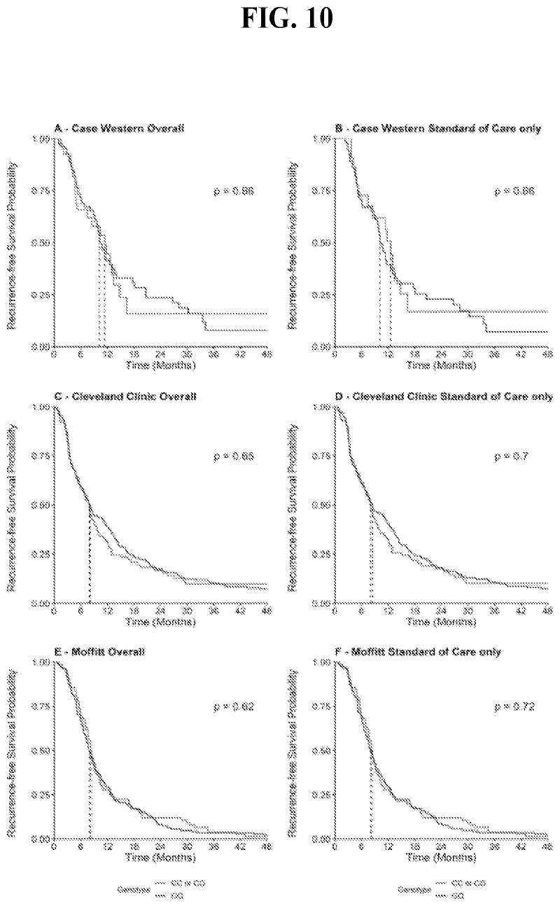

We assessed the rs755622 MIF SNP in three separate, annotated clinical cohorts of GBM patients (total of 966 patients including 449 from Cleveland Clinic, 386 from Moffitt Cancer Center, and 131 from Case Western Reserve University/University Hospitals of Cleveland). Our analysis of individual and combined cohort statistics revealed significant differences in the frequency of key prognostic indicators between cohorts (Karnofsky Performance Score (KPS), total surgical resection, receipt of standard of care (SOC), and recurrence) across the 3 cohorts. FIG. 7A-B). We genotyped all patients and found a similar frequency of MIF SNP rs755622 major (G/G) and minor allele-containing (C/C or C/G, noted as C/*) genotypes across cohorts (FIG. 7C) and when compared to the 1000 Genomes Project among those with European ancestry (FIG. 7C). Additionally, we found strong linkage disequilibrium between the rs755622 SNP and the CATT repeats rs5844572 (FIG. 8A,B) >90% co-segregation between CATT repeats 7-8 (rs5844572) and the MIF snp (rs755622), as previously reported⁴⁴. When evaluated according to major clinical prognostic indicators for GBM, we observed significant associations of sex and receipt of SOC with the rs755622 MIF SNP genotype using descriptive analysis (FIG. 7B, D, E, FIG. 9). Univariate and multivariable survival analyses did not demonstrate any differences in overall or progression-free survival according to rs75622 MIF SNP genotype in any individual cohort or when data from all 3 cohorts were combined (FIG. 7D-F; Supplemental FIG. 9, 10).

We observed no significant difference in GBM incidence or patient survival between the rs755622 MIF SNP genotypes, but we hypothesized that there may be differences in tumor and microenvironment interactions between genotypes given the association of the rs755622 MIF SNP with inflammation in non-oncologic conditions (Table 1).

TABLE 1
MIF SNP rs755622 highlighted literature in inflammatory associated conditions. Summary highlighting selected articles pertaining
to rs755622 history and research in inflammatory conditions, and provides relative clinical importance of this germline SNP.

| PMD | Title |
|---|---|
| PMD: 199416 [symbol] 1 | A MIF haplotype is associated with the outcome of patients with severe sepsis: a case control study |
| PMD: 20388 [symbol] 0 | Predictors of response to intra-articular steroid injection in psoriatic arthritis |
| PMD: 204476 [symbol] 8 | The MIF [symbol] 17 [symbol] G/C polymorphism and risk of childhood acute lymphoblastic leukemia in a Chinese population |
| PMD: 1638091 [symbol] | Replication of putative candidate-gene associations with rheumatoid arthritis in >4,000 samples from North America and Sweden: |
| | association of susceptibility with PTPN22, CTLA4, and PAD [symbol] |
| PMD: 175858 [symbol] 0 | Macrophage migration inhibitor factor in acute lung injury: expression, biomarker, and associations. |
| PMD: 20811 [symbol] 2 [symbol] | Genetic variants in inflammation related genes are associated with radiation-induced toxicity following treatment for non-small |
| | cell lung cancer. |
| PMD: 234027 [symbol] 2 | Macrophage migration inhibitory factor (MIF): genetic evidence for participation in early onset and early stage rheumatoid arthritis |
| PMD: 241 [symbol] 1398 | Macrophage migration inhibitory factor gene polymorphisms in inflammatory bowel disease: An association study in New Zealand |
| | Caucasians and meta-analysis |
| PMD: 2 [symbol] 167210 | Contribution of Macrophage Migration inhibitory Factor [symbol] 1730/C Gene Polymorphism [symbol] the Rist of Cancer in Chinese [symbol] |
| PMD: 265427 [symbol] 1 | Functional polymorphisms in the gene encoding macrophage migration inhibitory factor (MIF) are associated with active pulmonary |
| | [symbol] |
| PMD: 29545 [symbol] 22 | Macrophages Migration Inhibitory Factor [symbol] 173 [symbol] /C Polymorphism: A Global Meta-Analysis across the Disease Spectrum |
| PMD: 32 [symbol] 06 [symbol] | MIF [symbol] 173 [symbol] /C rs7555622) polymorphism modulates coronary artery disease risk: evidence from a systematic meta-analysis. |
| PMD: 2211 [symbol] 57 [symbol] | Polymorphisms in immune function genes and non-Hodgkin lymphoma survival |
| PMD: 24530749 | Macrophage migration inhibitory factor: association of [symbol] 73 [symbol] CA [symbol] 5-8 and [symbol] 173 G > C polymorphisms with TNF-α in systemic lupus |
| | [symbol] |
| PMD: 27 [symbol] 094 | A Macrophage Migration Inhibitory Factor Polymorphism is Associated with Autoimmune Hepatitis Severity in US and Japanese |
| | Patients |
| PMD: 2 [symbol] 81540 | MIF functional polymorphisms ( [symbol] 794 CATT 5-8 and [symbol] 173 G > C) are associated with MIF serum levels, severity and progression |
| | in [symbol] multiple sclerosis from western Mexican population |
| PMD: 2 [symbol] 0 [symbol] | MIF [symbol] 373G/C (rs755622) polymorphism as a risk factor for acute lymphoblastic leukemia development in children |
| PMD: 307473 [symbol] 2 | Macrophage migration inhibitory factor polymorphisms are a potential susceptibility marker in systemic sclerosis from southern |
| | Mexican population: association with MIF miRNA expression and cytokine profile |
| PMD: [symbol] 197 [symbol] 278 | Association of the genetic variants ( [symbol] 794 CATT [symbol] - [symbol] and [symbol] 173 G > C) of macrophage migration inhibitory factor (MIF) |
| | with higher [symbol] levels of MIF and INFα in women with breast cancer |

[symbol]

indicates data missing or illegible when filed

To explore this possibility, we selected 17 patients with primary, untreated GBM from each MIF genotype in our Cleveland Clinic cohort with similar clinical parameters and outcomes (Table 2) and subjected their tumor tissue to bulk RNA-sequencing.

TABLE 2

| Characteristic | CC, N = 1¹ | | CG, N = 16¹ | | GG, N = 17¹ | |
|---|---|---|---|---|---|---|
| race | | | | | | |
| black | 0 | (0%) | 1 | (6,2%) | 1 | (5.9%) |
| non hispanic white | 1 | (100%) | 15 | (94%) | 16 | (94%) |
| age | 59 | (59, 59) | 64 | (53, 69) | 59 | (49, 66) |
| sex | 1 | (100%) | 6 | (38%) | 6 | (35%) |
| kps.at.diagnosis | | | | | | |
| 60 | 0 | (0%) | 1 | (6.2%) | 0 | (0%) |
| 70 | 0 | (0%) | 1 | (6.2%) | 1 | (5.9%) |
| 80 | 0 | (0%) | 8 | (50%) | 4 | (24%) |
| 90 | 1 | (100%) | 6 | (38%) | 12 | (71%) |
| deletion_1p | 0 | (0%) | 1 | (6.2%) | 2 | (12%) |
| deletion_19q | 0 | (0%) | 5 | (31%) | 1 | (6.2%) |
| idh1_mutated | 0 | (0%) | 0 | (0%) | 1 | (5.9%) |
| mgmt_methylated | 0 | (NA %) | 2 | (33%) | 4 | (80%) |
| ki_67 | 5 | (5, 5) | 25 | (19, 32) | 25 | (14, 32) |
| egfr_amplified | 0 | (0%) | 9 | (56%) | 5 | (31%) |
| pfs | 19 | (19, 19) | 8 | (5, 21) | 25 | (5, 51) |
| os | 27 | (27, 27) | 12 | (10, 25) | 36 | (9, 82) |

¹n (%); Median (IQR)

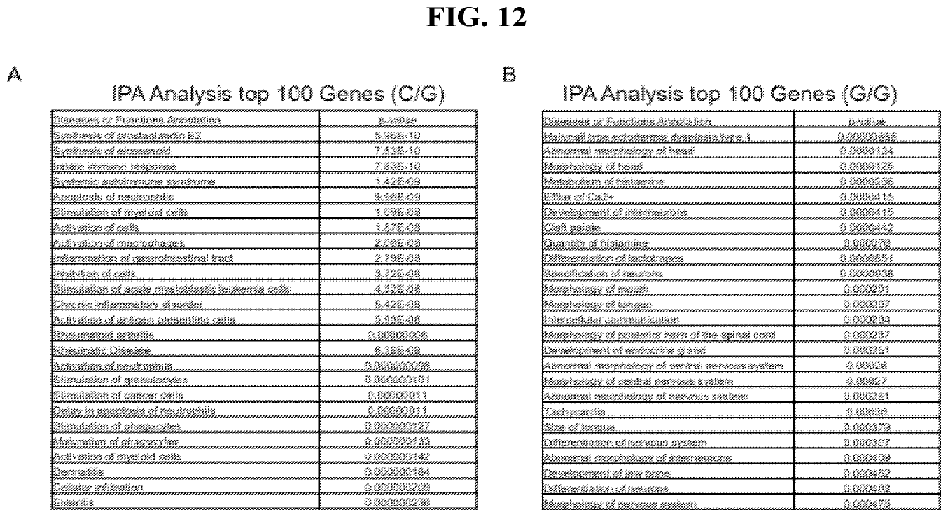

Differential gene expression analysis revealed that the rs755622 minor allele patients (e.g., −173 C SNP) had an enrichment in immune cell related genes (FIG. 1C, D). Gene set enrichment analysis (GSEA) utilizing the Hallmark curated gene sets identified a significant increase in inflammatory pathways in the minor allele patients (FIG. 1E). Additionally, ingenuity pathway analysis (IPA) showed similar findings, with increased innate immune response and prostaglandin signaling among the top enriched pathways of patients with the MIF SNP minor allele (FIG. 12). In seeking to better understand the individual immune cell types that were changed between genotypes, we used a deconvolution approach using ssGSEA with curated gene sets and found a significant increase in cells associated with an adaptive immune response in CG compared to GG patients (FIG. 1F). Of the differentially expressed genes, lactotransferrin (LTF) was the most significantly upregulated in patients with the minor allele and positively correlated with T cell and adaptive immune responses (FIG. 1G). LTF is considered a key factor in first line immune defense against bacteria, yeast, viruses, fungi, and parasites, and may additionally contribute to the anti-tumor response⁴⁵⁻⁴⁷ LTF is an acute phase immune mediator released from neutrophils and participates in the switch from innate to adaptive immune response. LTF signals through Toll-like receptors in myeloid cells to activate NFκB and CD40 expression and promote the initiation of anti-tumor immune responses^{48,49}. Taken together, these analyses suggest that while the rs755622 MIF SNP did not associate with differences in GBM incidence or survival, it did correlate with a difference in the immune cell composition within the tumor microenvironment.

Immunofluorescence Confirms Enhanced T Cell Infiltration and CD8 T Cell Activation in GBM Patients with the MIF SNP Rs755622

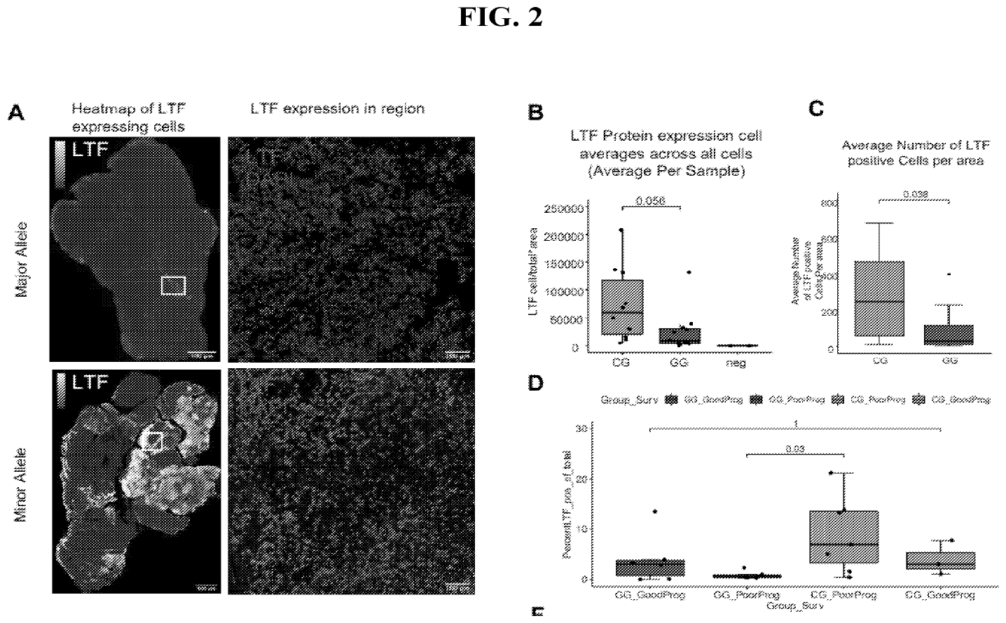

To further interrogate the immune cell differences between MIF genotypes, we utilized matched tissue from 22 patients (11 C/* and 11 G/G) from the same cohort subjected to RNA-sequencing. Staining for LTF confirmed the RNA-sequencing analysis and identified that LTF protein was increased in minor allele patients (FIG. 2A-C). The percent of LTF-expressing cells was further enhanced in minor allele patients with short term survival, based on an overall survival of less than the median of the 34 samples in this cohort (FIG. 2D). Additional assessment of T cell populations identified significant increases in CD8+ T cells in minor allele patients (FIG. 2E-F). Additionally, CD8+ T cells were less impacted by prognosis (FIG. 2G). While analysis of the activation marker CD107a on CD8+ T cells showed no difference between the good and poor prognosis groups (FIG. 2H), CD107a was significantly increased in CD8+ T cells in the minor allele patients (FIG. 2I). These data support the conclusion of an increase in immune infiltration of a cytotoxic T cell population in the minor allele patients along with enhanced LTF expression in the tumor microenvironment.

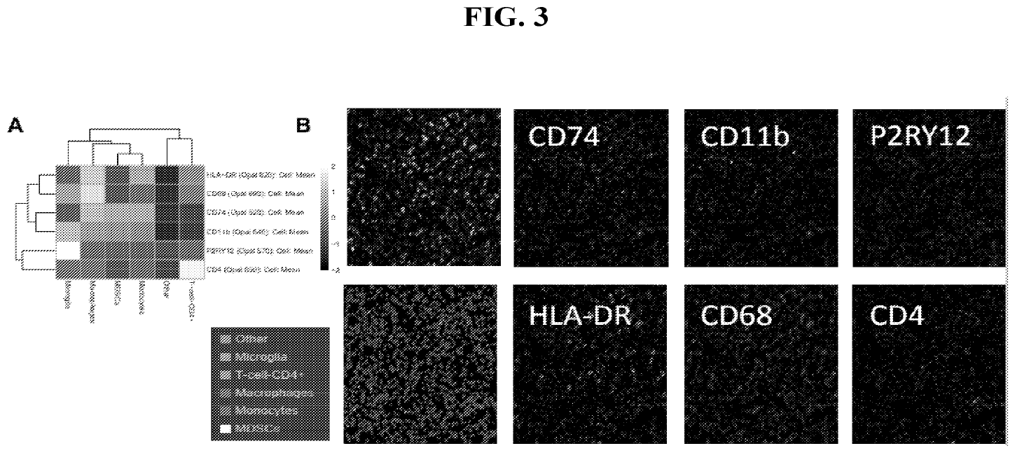

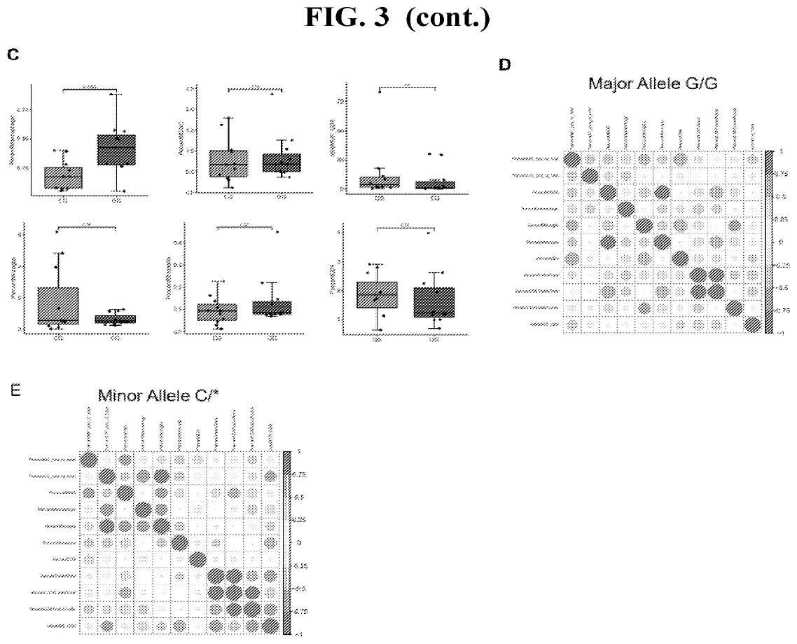

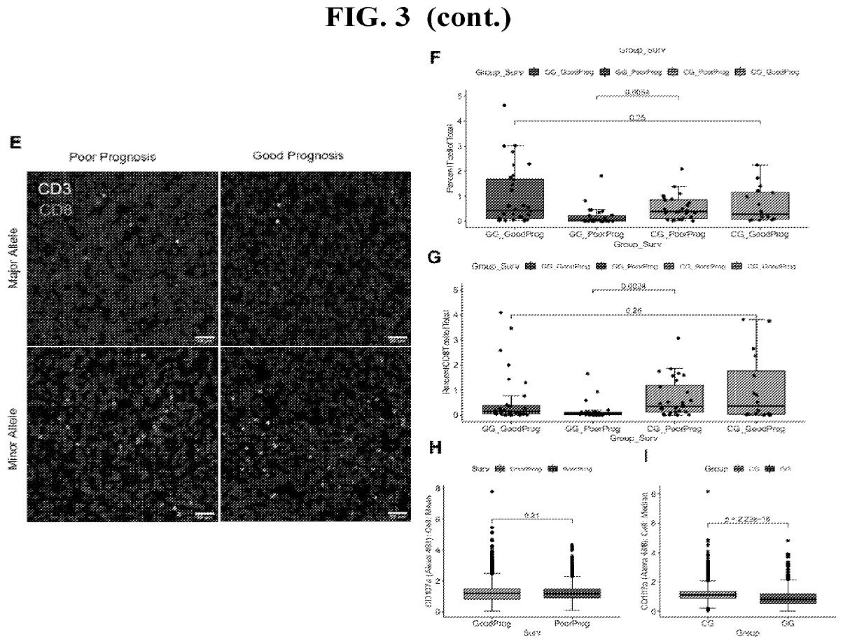

While we observed changes in the lymphoid compartment from the RNA-sequencing studies and validated these in human patient samples, these initial assessments did not focus on specific myeloid cell subtypes that are known to be involved in GBM immune suppression (including microglia, monocytes, macrophages, and MDSCs). Using matched samples from the RNA sequencing study, we next stained for CD4+ T cells and for myeloid cell subtypes known to be involved in GBM immune suppression (e.g., microglia, monocytes, macrophages, and MDSCs (n=11 C/* and n=11 G/G). Individual cell subtypes were identified using the top quartile for lineage-specific markers (CD4+ T cells: CD4⁺, CD11b⁻; macrophage: CD11b⁺, CD68⁺, HLA-DR⁺; microglia: P2RY12⁺; MDSCs: CD11b⁺, CD74⁺, CD68⁻, HLA-DR⁻; monocytes: CD11b⁺, HLA-DRA⁺, P2RY12⁻, CD68⁻) (FIG. 3A, B). Quantification revealed decreased macrophages in minor allele patients but no other major changes in MDSCs, ratio of MDSCs to CD8+ T cells, microglia, monocytes, and CD4+ T cells between genotypes (FIG. 3C). Due to the quantitative difficulties of assessing all myeloid cell lineages in an individual panel with lymphoid populations, we correlated cell types across samples per genotype (FIG. 3D, E). Using this approach, we found striking differences between genotypes, with the minor allele patients exhibiting an increase in CD8+ T cells and reductions in the myeloid compartment. Additionally, we found that LTF positively correlated with microglia and negatively correlated with the ratio of MDSCs to CD8+ T cells. These data lead to the overall conclusion that minor allele patients have increased lymphocyte infiltration with reduced macrophage content and further that LTF is associated with increased CD8+ T cells.

GBM Patients with High LTF Expression are Immunologically Activated

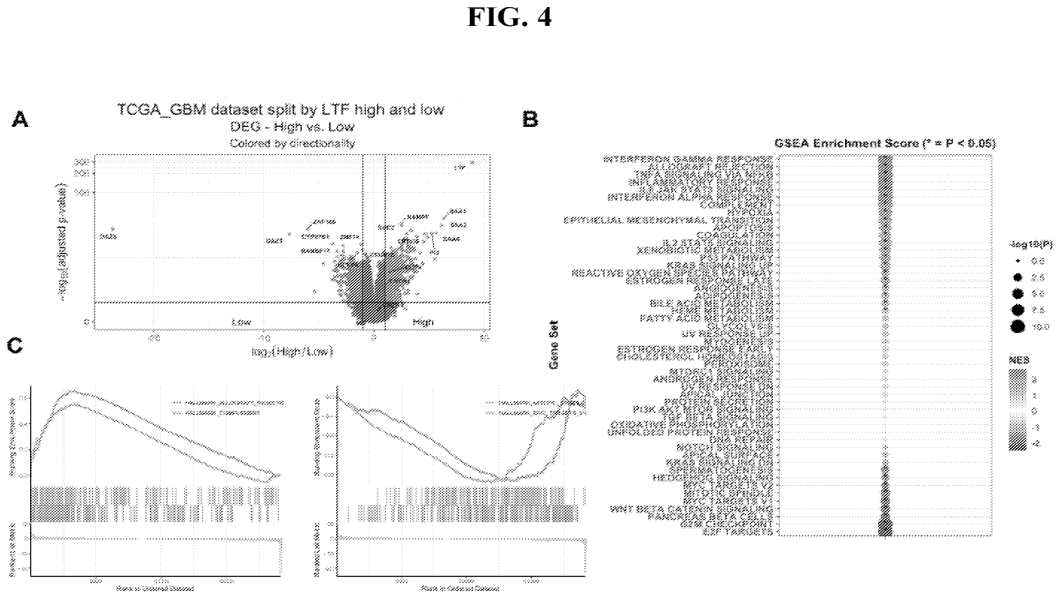

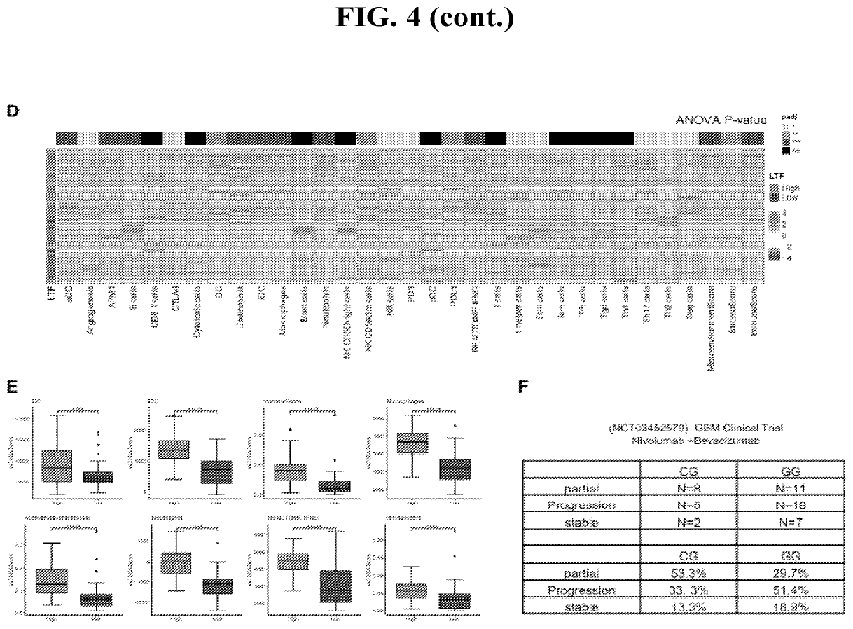

Seeking to expand on these initial observations, we sought to identify the rs755622 SNP in The Cancer Genome Atlas (TCGA) dataset but were unsuccessful because this marker is too far upstream of transcription to have read coverage in whole exome sequencing data. Given the strong correlation between the MIF minor allele with LTF expression, we used LTF expression as a surrogate of MIF genotype and interrogated immune changes associated with LTF. Notably, we found a similar differential expression profile between LTF-high (top 25%) and LTF-low (bottom 25%) patients in the TCGA as we did between genotypes in our dataset (FIG. 4A). In agreement with our initial assessment, GSEA revealed an increase in immune activation pathways in the LTF high expression patients, including allograft rejection and complement signaling, and a reduction in cancer-related pathways, including mitotic spindle and myc targets (FIG. 4B, C). To further identify cell type estimates between LTF-high and LTF-low samples, we performed deconvolution analysis and found increased immune and microenvironment scores, interferon gamma score, and macrophage content (FIG. 4D, E).

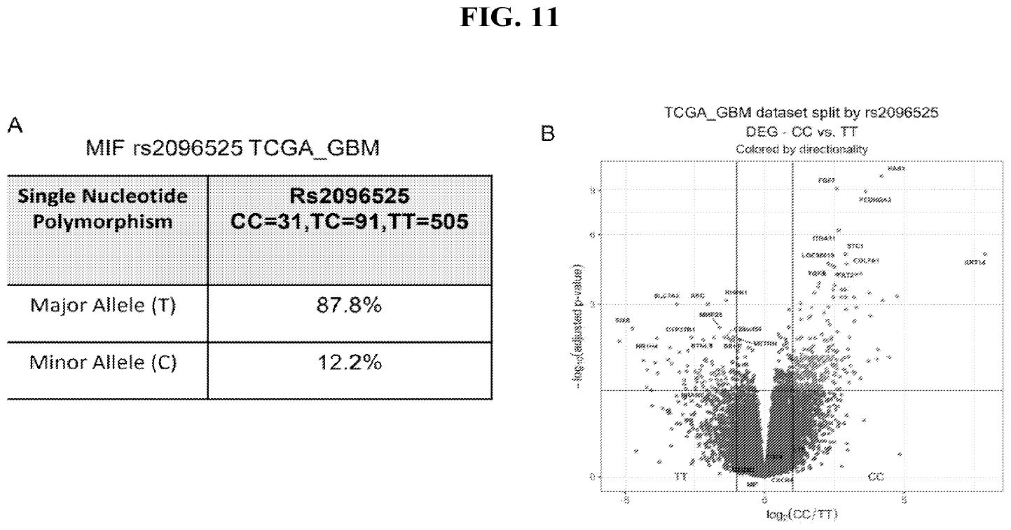

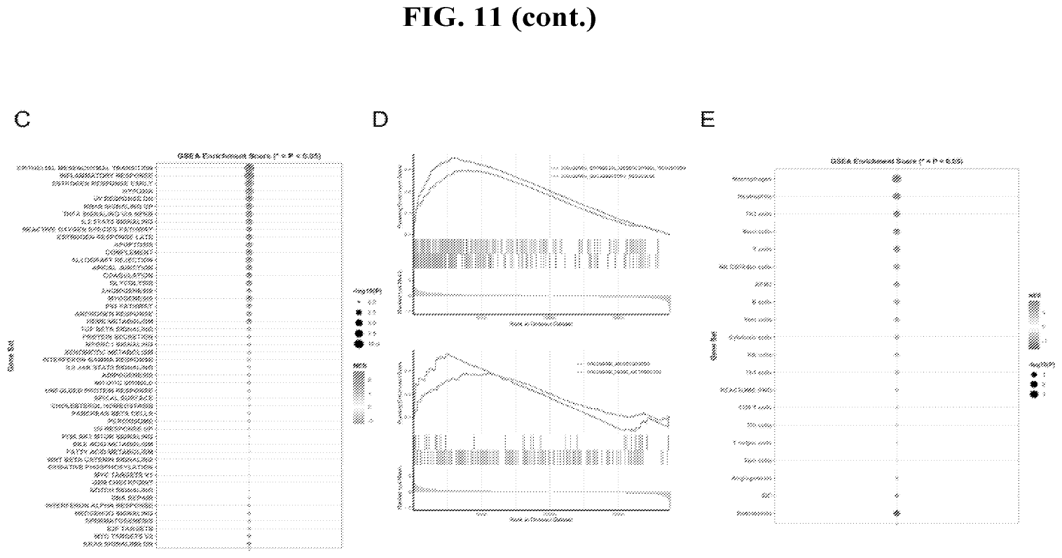

To determine whether these findings with respect to LTF signature recapitulate the rs755622 genotype, we examined possible associations with the rs2096525 SNP, which is in linkage disequilibrium with rs755622^{50,51} but located within the first or second MIF intron. We interrogated the TCGA GBM whole exome sequencing dataset for the rs2096525 SNP and we found the minor allele to be present in approximately 12% of GBM patients (FIG. 11A). A deconvolution analysis showed increased inflammatory response pathways and similar increases in macrophages, neutrophils, and T cells for this SNP as observed for rs755622. We therefore hypothesized on the basis of these findings that there could be a differential response to immunotherapy based on the MIF genotype. We accessed data from an ongoing clinical trial of nivolumab in recurrent GBM patients (NCT03452579) and found that MIF minor allele patients (e.g., rs). showed a stronger response to immune checkpoint inhibitors (FIG. 4F).

### LTF Predicts Immunotherapy Response

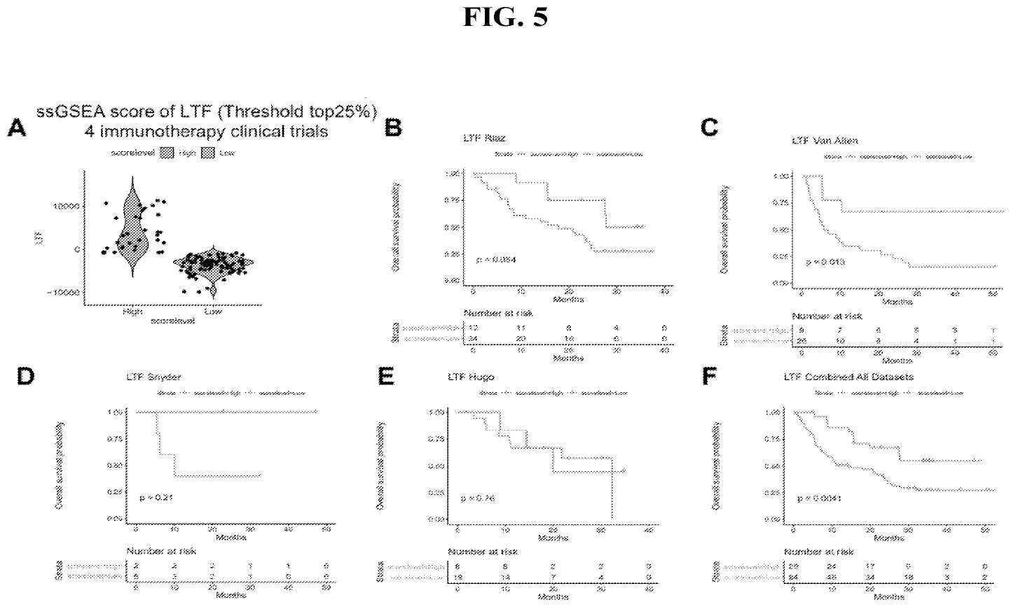

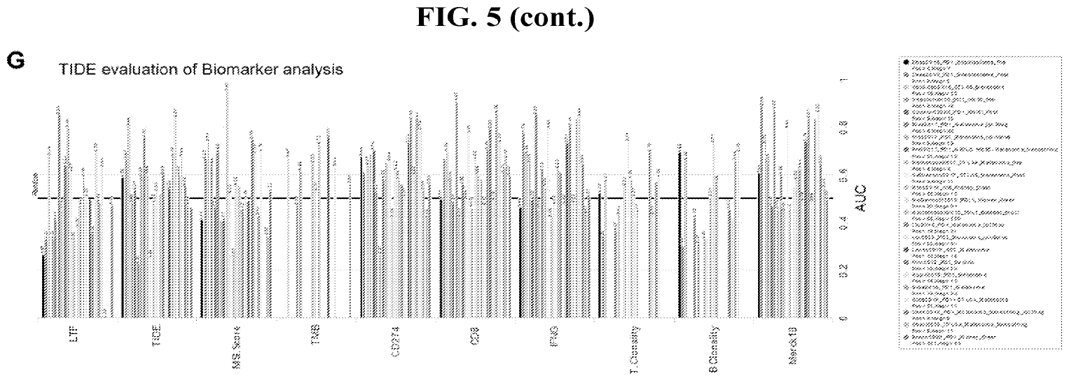

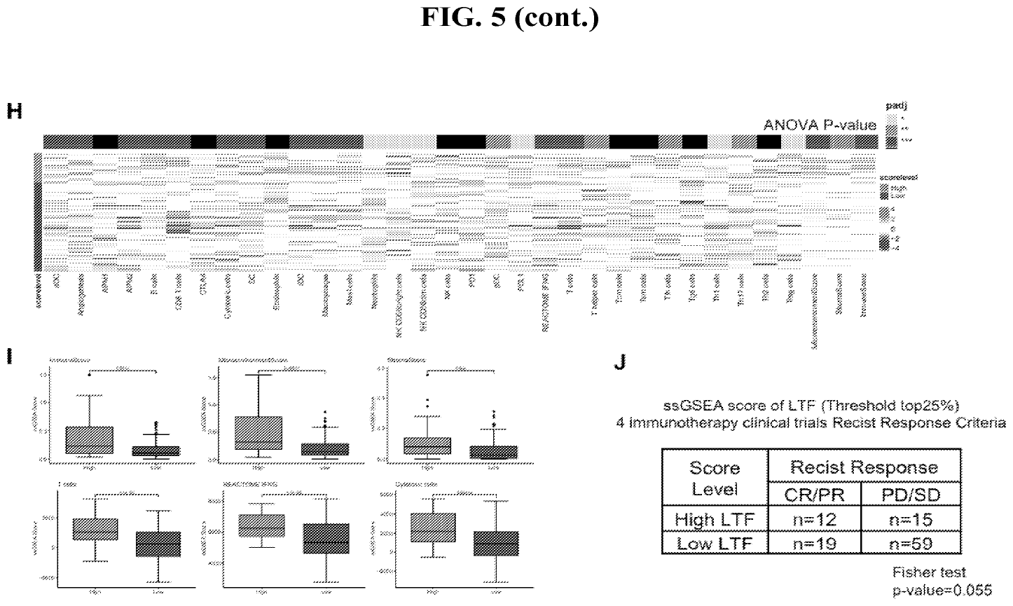

Our queried GBM datasets are limited by the circumstance that overall clinical responses are weaker than studies of other solid tumors, including melanoma and non-small cell lung cancer. To determine whether the influence of MIF genotype may extend beyond GBM, we interrogated four melanoma immunotherapy clinical trials with available RNA-sequencing data. We separated high LTF-expressing patients based on the top 25% of expression (FIG. 5A). Utilizing these categories, we found an increase in overall survival in three out of the four datasets (FIG. 5B-D). When combined, we observed a robust difference in survival across all four datasets (FIG. 5F). To further evaluate LTF as a biomarker of immunotherapy response, we utilized the tumor immune dysfunction and exclusion (TIDE) platform and observed that across 23 immunotherapy datasets, LTF demonstrated an area under the curve greater than random in 9 of the datasets (FIG. 5G). As shown this is similar to other known measures of response, including CD8 and interferon gamma expression using TIDE (FIG. 5G).

To determine the cellular composition changes that occur in the LTF high vs low immunotherapy patients we utilized deconvolition analyses. Deconvolution analysis of the melanoma cohorts analyzed in univariate analyses for FIG. 5A-F identified an enrichment of immune cell signatures p<0.05 for ANOVA tests in 25/34 cell types and scores tested (FIG. 5H). Specifically, ImmuneScore, MicroenvironmentScore, StromaScore, T cells, Reactome IFNG, and cytotoxic cells were enriched (FIG. 5I). Lastly, using the Recist criteria provided from each publication^{7,37,39,40} LTF high and low samples were separated into response categories, complete responders/partial responders (CR/PR) and progressive disease/stable disease (PD/SD), and then a fisher test was used to evaluate the difference in patient distribution. In this analysis the Fisher test (p=0.055) (FIG. 5J) demonstrated a strong trend toward differences in response rates between high and low LTF groups similar to our observations in the GBM immunotherapy clinical trial (FIG. 4F). Taken together, our analysis reveals that elevated LTF expression is correlated with immunotherapy response and changes in the immune microenvironment, which are also similar to GBM patients with the MIF minor allele genotype.

MIF, considered the first cytokine activity⁵², has been extensively studied in the context of immune activation and the inflammatory response, as well as in tumor biology, where it has been shown to drive cancer cell proliferation and the generation of a tumor-promoting immune microenvironment⁵³. In GBM, MIF functions include enhancement of CSC maintenance^{54,55}, resistance to therapies including standard-of-care therapy temozolomide⁵⁶ as well as the anti-angiogenic agent bevacizumab⁵⁷, as well as altering growth factor receptor signaling⁴⁸. However, the potential functional consequences of common MIF promoter polymorphisms, such as the −173 SNP (rs755622), have not been well studied in inflammation associated with malignancies. While the rs755622 SNP is associated with numerous inflammatory conditions and certain cancers, particularly those sensitive to immunotherapies^{19,53,58,59}, we found that in GBM, there was no correlation with incidence or prognosis in response to standard-of-care therapy across three studied cross-institutional cohorts. This finding extended to the functional MIF promoter −794 CATT microsatellite (rs5844572) that is in linkage disequilibrium with the rs755622 SNP. We also observed no major difference in MIF level between genotypes, likely due to the elevated level of MIF in GBM and is further confound by many GBM patients being treated with dexamethasone, which increased MIF production⁶⁰. However, we found evidence for distinct tumor immune microenvironment between genotypes, with a heightened increase in CD8+ T cells in the minor allele patients. This enhancement in immune response parameters correlated with an enhancement in LTF expression in the minor allele patients, which further predicted immunotherapy response in GBM patients as well as those with melanoma and non-small cell lung cancer.

Our initial assessment of MIF genotypes revealed an association between the minor SNP allele and LTF, which has not been previously described. In non-pathophysiological conditions, LTF is an iron binding glycoprotein that functions to protect against pathogens and has been shown to have anti-inflammatory activity. LTF has been described in cancer to function in an anti-proliferative manner. In GBM, LTF expression is reduced compared to lower-grade brain tumors⁶¹ and can inhibit GBM cell proliferation⁶². LTF also has also been studied as a nanoparticle carrier for a variety of pre-clinical cancer therapies, including in GBM, where it has brain penetrance⁶³. In the context of MDSCs, we found that MIF enhances MDSC function in GBM and that LTF can induce MDSCs in pathological neonatal inflammatory conditions⁶⁴.

Utilizing both RNA-sequencing and matched tissue samples, we identified that MIF minor SNP allele patients who had increased LTF expression, also had an increase in CD8+ T cells and a reduction in macrophages, with no change in MDSCs. We also not observe a consistent change in tumor associated macrophages between MIF SNP patients and LTF expression, which could be due to a limitation of deconvolution methods in distinguishing myeloid subtypes. However, taken together, this immune microenvironment is likely more conductive to immune-activating strategies, and this was confirmed in ongoing data from a small clinical trial in GBM and multiple melanoma clinical trials. The utility of LTF as a marker of a more inflammatory microenvironment with potential consequences for immunotherapy response was further demonstrated with the TIDE database platform. While our initial analysis revealed a high correlation between the MIF minor allele and elevated LTF, the association between the MIF SNP minor allele and LTF was made indirectly, due to the inability to efficiently identify the MIF SNP status in large genomic datasets based on its location in the promoter region, which is not covered by whole exome sequencing. Nonetheless, utilizing LTF at a median cut-off does not yield the same results as the top quartile of LTF, which more closely represents the frequency of the MIF SNP minor allele. Another limitation of our findings is that the MIF SNP has not been functionally characterized but is in linkage disequilibrium with the MIF −794 CATT repeat, which is associated with an increase in MIF production in immune cells and brain tissues.

The known genetic determinants of immunotherapy response in gliomas are currently limited to somatic mutations in IDH^{65,66}, and the present findings identify a common germline SNP with clear immunologic significance that may be utilized to improve clinical decision-making and support development of more effective immunotherapies.

### REFERENCES

1 Brahmer, J. et al. Nivolumab versus Docetaxel in Advanced Squamous-Cell Non-Small-Cell Lung Cancer. N Engl J Med 373, 123-135, (2015).
2 Motzer, R. J. et al. N Engl J Med 373, 1803-1813, (2015).
3 Robert, C. et al. N Engl J Med 372, 2521-2532, (2015).
4 Yao, J. et al. J Clin Invest 126, 732-744, (2016).
5 Weenink, B., et al., Cancers (Basel) 12, (2020).
6 Reardon, D. A. et al. Effect of Nivolumab vs Bevacizumab in Patients With Recurrent Glioblastoma: The CheckMate 143 Phase 3 Randomized Clinical Trial. JAMA Oncol, doi:10.1001/jamaoncol.2020.1024 (2020).
7 Cloughesy, T. F. et al. Nat Med 25, 477-486, (2019).
8 Alban. T. J. et al. Frontiers in Immunology 11, doi:10.3389/fimmu.2020.01191 (2020).
9 Bayik, D. et al. Myeloid-derived suppressor cell subsets drive glioblastoma growth in a sex-specific manner. Cancer Discov, doi:10.1158/2159-8290.CD-19-1355 (2020).
10 Bayik, D. & Lathia, J. D. Nat Rev Cancer 21, 526-536, (2021).
11 Peereboom, D. M. et al. Metronomic capecitabine as an immune modulator in glioblastoma patients reduces myeloid-derived suppressor cells. JCI Insight 4, doi:10.1172/jci.insight.130748 (2019).
12 Alban, T. J. et al. Global immune fingerprinting in glioblastoma patient peripheral blood reveals immune-suppression signatures associated with prognosis. JCI Insight 3, doi:10.1172/jci.insight.122264 (2018).
13 Otvos, B. et al. Stem Cells 34, 2026-2039, (2016).
14 Alban. T. J. et al. Front Immunol 11, 1191, (2020).
15 Lehmann, L. E. et al. A MIF haplotype is associated with the outcome of patients with severe sepsis: a case control study. J Transl Med 7, 100, doi:10. 1186/1479-5876-7-100 (2009).
16 Xue, Y. et al. Leuk Res 34, 1282-1286, (2010).
17 Eder, L. et al. Rheumatology (Oxford) 49, 1367-1373, (2010).
18 Gao. L. et al. Transl Res 150, 18-29, (2007).
19 Hildebrandt. M. A. et al. Genetic variants in inflammation-related genes are associated with radiation-induced toxicity following treatment for non-small cell lung cancer. PLoS One 5, e12402, doi:10.1371/journal.pone.0012402 (2010).
20 Llamas-Covarrubias, M. A. et al. Cytokine 61, 759-765, (2013).
21 Falvey, J. D. et al. World J Gastroenterol 19, 6656-6664, (2013).
22 Wang, C. D., et al., Asian Pac J Cancer Prev 16, 4597-4601, (2015).
23 Kuai, S. G. et al. Infect Dis (Lond) 48, 222-228, (2016).
24 Illescas, et al., Macrophage Migration Inhibitory Factor −173 G/C Polymorphism: A Global Meta-Analysis across the Disease Spectrum. Front Genet 9, 55, doi:10.3389/fgene.2018.00055 (2018).
25 Li, D. Y. et al. MIF −173G/C (rs755622) polymorphism modulates coronary artery disease risk: evidence from a systematic meta-analysis. BMC Cardiovasc Disord 20, 300, doi:10.1186/s12872-020-01564-4 (2020).
26 Aschebrook-Kilfoy, B. et al. J Cancer Surviv 6, 102-114, (2012).
27 De la Cruz-Mosso, U. et al., Hum Immunol 75, 433-439, (2014).
28 Assis, D. N. et al., Dig Dis Sci 61, 3506-3512 (2016).
29 Castaneda-Moreno, et al, J Neuroimmunol 320, 117-124 (2018).
30 Sharaf-Eldein, M., Elghannam, D. & Abdel-Malak, C. MIF-173G/C (rs755622) polymorphism as a risk factor for acute lymphoblastic leukemia development in children. J Gene Med 20, e3044, doi:10.1002/jgm.3044 (2018).
31 Banos-Hemandez, C. J. et al. Clin Rheumatol 38, 1643-1654 (2019).
32 Avalos-Navarro, G. et al. Association of the genetic variants (−794 CATT₅-8 and −173 G>C) of macrophage migration inhibitory factor (MIF) with higher soluble levels of MIF and TNFalpha in women with breast cancer. J Clin Lab Anal 34, e23209, (2020).
33 Melin, B. S. et al. Nat Genet 49, 789-794, (2017).
34 Hanzelmann, S., Castelo, R. & Guinney, J. GSVA: gene set variation analysis for microarray and RNA-seq data. BMC Bioinformatics 14, 7. doi:10,1186/1471-2105-14-7 (2013).
35 Aran, D., Hu, Z. & Butte, A. J. xCell: digitally portraying the tissue cellular heterogeneity landscape. Genome Biol 18, 220, doi:10.1186/s13059-017-1349-1 (2017).
36 Jiang, P. et al. Nat Med 24, 1550-1558, (2018).
37 Rizvi, N. A. et al. Science 348, 124-128, (2015).
38 Hugo, W. et al. Cell 165, 35-44 (2016).
39 Riaz, N. et al. Cell 171, 934-949 e916, (2017).
40 Snyder, A. et al. N Engl J Med 371, 2189-2199, (2014).
41 Bankhead. P. et al. Sci Rep 7, 16878, (2017).
42 Uwe Schmidt, M. W., Coleman Broaddus, and Gene Myers. Cell Detection with Star-convex Polygons. International Conference on Medical Image Computing and Computer-Assisted Intervention (MICCAI) (2018).
43 Viratham Pulsawatdi, A. et al. Mol Oncol 14. 2384-2402, (2020).
44 Leng, L., Siu, E. & Bucala, R. Methods Mol Biol 2080, 67-84, (2020).
45 Rodriguez-Franco, et al., Rev Latinoam Microbiol 47, 102-111 (2005).
46 Yamauchi, K., et al., Biochem Cell Biol 84, 291-296, (2006).
47 Gonzalez-Chavez, et al., Int J Antimicrob Agents 33, 301 e301-308 (2009).
48 Zheng, Y. et al. Lab Invest 94, 1188-1199, (2014).
49 Curran, C. S., et al., Cell Immunol 242, 23-30, (2006).
50 Du, G. L. et al. MIF gene rs755622 polymorphism positively associated with acute coronary syndrome in Chinese Han population: case-control study. Sci Rep 10, 140, doi:10.1038/s41598-019-56949-z (2020).
51 Luo, J. Y. et al. Medicine (Baltimore) 95, e2617 (2016).
52 Kang, I. & Bucala, R. Nat Rev Rheumatol 15, 427-437 (2019).
53 Noe. J. T. & Mitchell, R. A. MIF-Dependent Control of Tumor Immunity. Front Immunol 11, 609948 (2020).
54 Meyer-Siegler, K. L. et al. Genes Immun 8, 646-652, (2007).
55 Donnelly, R. P. et al. J Immunol 193, 4477-4484, (2014).
56 Tuccari, et al., Eur J Histochem 43, 317-322 (1999).
57 Arcella, A. et al. J Neurosurg 123, 1026-1035 (2015).
58 Kumari, et al., Sci Rep 7, 6602, (2017).
59 Liu, Y. et al. J Clin Invest 129, 4261-4275, doi:10.1172/JCI128164 (2019).
60 Platten, M. et al. A vaccine targeting mutant IDH1 in newly diagnosed glioma. Nature 592, 463-468, (2021).
61 Ghouzlani, A. et al. Immune Checkpoint Inhibitors in Human Glioma Microenvironment. Front Immunol 12, 679425, (2021).
62. Baugh, Nature, Genes & Immunity, 3, 170176, 2002.
63. U.S. Pat. No. 7,745,121
64. Santoscoy-Ascencio et al., Mol Genet Genomic Med; 8(1):e1037,Epub 2019 November 7.
65. De-Yang et al., BMC Cardiovascular Disorders, 20, 300, 2020.
66. Morales-Zambrano et al., Int J Clin Exp Med.; 7(9):2605-14, 2014.
67. Association of genetic polymorphisms in MIF with breast cancer risk in Chinese women
Shuai Lin, Clin Exp Med. 17(3):395-401, Epub 2016
68. Suhui Wu, et al., J Obstet Gynaecol Res.; 43(3):571579, 2017.
69. Katia Mangano, et al, Oncotarget. 9:17951-17970, 2018
70. Xiao-bing Wang, et al., Journal of NeuroOncology volume 106, pages 43-51, 2012.
71. Ha W, et al., Sci Rep., 9(1):2905. 2019
72. Mario Presti, et al., Oncol Lett. 16(3): 2881-2886, 2018
73. Balint Otvos, et al., Neuro-Oncology 21(Supplement_6):vi103-Nov. 11, 2019
74. Balint Otvos et al., Society of NeuroOncology, Oral presentation, Phoenix, Arizona., Nov. 24, 2019.
75. Study to Evaluate Ibudilast and TMZ Combo Treatment in Newly Diagnosed and Recurrent Glioblastoma, MediciNova NIH National Library of Medicine, Dec. 20, 2018, NCT03782415.

All publications and patents mentioned in the specification and/or listed below are herein incorporated by reference. Various modifications and variations of the described method and system of the invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention that are obvious to those skilled in the relevant fields are intended to be within the scope described herein.

We claim:

1. A method performing an activity based on the presence of at least one polymorphism in the DNA of a patient with glioblastoma comprising:
a) performing a nucleic acid detection assay on a DNA sample from a subject, or receiving results from said assay,
wherein said assay detects the presence in the MIF promoter region of at least one polymorphism selected from: −173C and −794 CATT₅₋₈, and
wherein said subject has symptoms of glioblastoma; and
b) performing at least one of the following activities:
i) treating said subject with: a glioblastoma therapeutic agent, an immune modulating therapy for glioblastoma, pembrolizumab, ipilimumab, nivolumab, a viral therapy for glioblastoma, or a CAR-T cell therapy for glioblastoma;
ii) generating and/or transmitting a report that indicates the presence of said at least one polymorphism and that said subject has increased recurrence risk and/or more rapid decline in KPS status risk and/or decreased survival risk compared to glioblastoma patient's without one or both of said polymorphisms; and
iii) generating and/or transmitting a report that indicates the presence of said at least one polymorphism, and that said subject should be treated with a glioblastoma therapeutic agent, an immune modulating therapy for glioblastoma, pembrolizumab, ipilimumab, nivolumab, a viral therapy for glioblastoma, or a CAR-T cell therapy for glioblastoma.

2. The method of claim 1, wherein said subject's genotype is determined to be −173G/C or −173C/C.

3. The method of claim 1, wherein said subject's genotype is determined to be −794 CATT₅₋₈/CATT₄ or CATT₅₋₈/CATT₅₋₈.

4. The method of claim 1, wherein said subject has both of said polymorphisms.

5. The method of claim 1, wherein said report indicates that said subject has both of said polymorphisms.

6. The method of claim 1, wherein said −794 CATT₅₋₈ is −794 CATT₇.

7. The method of claim 1, wherein said detecting is conducted by a method comprising sequencing.

8. The method of claim 1, wherein step a) is receiving results from said assay, and wherein said at least one of the following activities is treating said subject.

9. The method of claim 8, wherein said treating is with said immune modulating therapy for glioblastoma.

10. The method of claim 8, wherein said treating is with said CAR-T cell therapy for glioblastoma.

11. A method for performing an activity based on elevated levels of Lactotransferrin (LTF) in a biological sample from a patient with cancer comprising:
a) performing a detection assay on a biological sample from a subject, or receiving results from said assay,
wherein said assay detects an increased level of Lactotransferrin (LTF) mRNA and/or LTF protein compared to control levels, and
wherein said subject has symptoms of cancer; and
b) performing at least one of the following activities:
i) treating said subject with: an immune modulating therapy, pembrolizumab, ipilimumab, nivolumab, or a CAR-T cell therapy;
ii) generating and/or transmitting a report that indicates the increased level of LTF mRNA and/or protein and that said subject should be treated with an immune modulating therapy, pembrolizumab, ipilimumab, nivolumab, or a CAR-T cell therapy.

12. The method of claim 11, wherein said cancer is glioblastoma.

13. The method of claim 11, wherein said cancer is melanoma.

14. The method of claim 11, wherein said LTF mRNA or LTF protein is said LTF mRNA.

15. The method of claim 11, wherein said LTF mRNA or LTF protein is said LTF protein.

16. The method of claim 11, wherein step a) is receiving results from said assay, and wherein said at least one of the following activities is treating said subject.

17. The method of claim 16, wherein said treating is with said immune modulating therapy.

18. The method of claim 17, wherein said immune modulating therapy is immune modulating therapy for glioblastoma or melanoma.

19. The method of claim 16, wherein said treating is with said CAR-T cell therapy.

20. The method of claim 19, wherein said CAR-T cell therapy is CAR-T cell therapy for glioblastoma or melanoma.


SEQUENCE LISTING (7 sequences)

SEQ ID NO: 1 - DNA, 18 bp, Artificial sequence
  Primer
cccaaagacaggaggtac

SEQ ID NO: 2 - DNA, 19 bp, Artificial sequence
  Primer
atgatggcagaaggaccag

SEQ ID NO: 3 - DNA, 20 bp, Homo sapiens
cattcattcattcattcatt

SEQ ID NO: 4 - DNA, 24 bp, Homo sapiens
cattcattcattcattcattcatt

SEQ ID NO: 5 - DNA, 28 bp, Homo sapiens
cattcattcattcattcattcattcatt

SEQ ID NO: 6 - DNA, 32 bp, Homo sapiens
cattcattcattcattcattcattcattcatt

SEQ ID NO: 7 - DNA, 16 bp, Homo sapiens
cattcattcattcatt